(19) United States
(12) Patent Application Publication
(10) Pub. No.: US 2023/0381025 A1
(43) Pub. Date: Nov. 30, 2023

(54) SITUATIONAL AWARENESS, COMMUNICATION, AND SAFETY IN HEARING PROTECTION AND COMMUNICATION SYSTEMS

(71) Applicant: PICOTERA ELECTRONICS INC., Richmond (CA)

(72) Inventors: Kamyar Keikhosravy, Richmond (CA); Kenneth Norman Trudel, Edmonton (CA)

(21) Appl. No.: 18/366,590

(22) Filed: Aug. 7, 2023

Related U.S. Application Data
(63) Continuation-in-part of application No. 17/177,929, filed on Feb. 17, 2021, now Pat. No. 11,722,813.

(51) Int. Cl. A61F 11/14; A61F 11/08; H04W 64/00; H04B 17/327

(52) U.S. Cl. CPC A61F 11/14; H04B 17/327; H04W 64/003; A61F 11/08

(57) ABSTRACT

An apparatus for hearing protection comprises a pair of earpads, a band, microphones, speakers, vibration generators, and a processing unit. Each of the earpads is placed over an ear of the user. The band extends between the pair of earpads. The microphones convert acoustic signals into electrical signals. The speakers are located on each of the pair of earpads and direct sound towards the ear. The vibration generators are located on at least one of the pair of earpads and generate vibratory feedback to the user. The processing unit is connected to the microphones, the speakers, and the vibration generators, and compares first parameters of the electrical signals from the microphones with second parameters of predetermined warning sounds to determine whether the electrical signals comprise one or more of the predetermined warning sounds. If the processing unit determines that the electrical signals comprise one or more of the predetermined warning sounds, the processing unit transmits a warning to the speakers and the vibration generators.

### FIELD OF THE INVENTION

The present invention relates to hearing protection and communications devices and systems. In particular, the invention relates to methods and apparatuses for improving the situational and directional awareness and communicational ability of users wearing hearing protection devices.

### BACKGROUND OF THE INVENTION

Noise at industrial sites is a significant cause of work-related accidents and injuries.

Conventional hearing protection devices (HPD), such as headphones, earmuffs, or earplugs, provide some degree of acoustic protection. This may be through passive noise reduction (which involves using insulating materials to block sound from the ears) or active noise reduction (which involves electronic circuitry to generate noise in negative phase to cancel out unwanted external noise).

One of the problems with conventional HPDs is that the user wearing such devices will often have lower situational and directional awareness and have a harder time communicating with other users. This is because the noise reduction offered by HPDs will also have the effect of attenuating both unwanted and wanted sounds. Therefore, users may miss or not hear sounds that are important (e.g. warning sound and alarms, spoken words, etc.). Furthermore, even if the user hears a warning sound or alarm, the user may not be aware that the warning sound or alarm is for them and/or that there is any danger and therefore ignore the warning.

Acoustic warning detectors disclosed in the prior art typically comprise complex building blocks with high power and processing requirements. The prevalence of portable music players accompanied with high-quality noise cancellation headphones may allow for simpler warning detection systems. Such a system has to detect some or all types of acoustic warning signals, such as police and ambulance sirens, car and truck's horns, loud noises, designated words, etc. Upon detection of any of such warning signals, the system should generate a configurable notification for the user. The system should also be energy-efficient, portable, configurable, and capable of communicating with popular music players in the market.

It is therefore desirable to provide a HPD that also allows for improved situational and directional awareness by utilizing a combination of warning sound detection, noise cancellation, built-in communication and vocal sound amplification. It is also desirable that such a HPD allow for communications among users and remote monitoring.

### SUMMARY OF THE INVENTION

The present invention comprises a hearing protection device that is configured to detect and identify predetermined warning sounds and signals. The predetermined warning sounds can be static (e.g. configured at factory) or alternatively they may be updated by the users in-situ. Upon detection of such warning sounds or signals, a notification may be provided to the user.

The hearing protection device also provides adjustable acoustic protection, which is able to automatically (or manually by the user) adjust the level of acoustic protection based on external noise levels measured in real-time in a hearing protection device or another device connected to the hearing protection device. Based on a real-time calculated surrounding noise level, the hearing protection device is configured to increase or decrease the acoustic protection by adjusting the volume of environmental sounds that is provided to the user's ears.

In another aspect of the invention, the hearing protection device is configured to provide for wireless communications with other hearing protection devices. In addition, the hearing protection device may be paired with a wristband or other wearable devices, such as smart watches or smart goggles (e.g. with heads-up displays). Alternatively, it may be paired to communicate with a central monitoring system.

In yet another aspect of the invention, the hearing protection device may be connected to a mobile device. Communications between different users may be achieved through communication between the respective mobile devices.

In a further aspect of the invention, the hearing protection device may be configured to detect tap patterns, which may be used to issue various commands to the hearing protection device.

In one aspect of the invention, an apparatus for hearing protection for a user comprises a pair of earpads, a band, one or more microphones, one or more speakers, one or more vibration generators, and a processing unit. Each of the earpads is adapted to be placed over an ear of the user and comprises an exterior surface. The band extends between the pair of earpads. The microphones are adapted to convert acoustic signals into electrical signals, with the microphones located on the exterior surface. The speakers are located on each of the pair of earpads and are adapted to direct sound towards the ear. The vibration generators are located on at least one of the pair of earpads and are adapted to generate vibratory feedback to the user. The processing unit is connected to the microphones, the speakers, and the vibration generators, and the processing unit is adapted to compare first parameters of the electrical signals from the microphones with second parameters of predetermined warning sounds to determine whether the electrical signals comprise one or more of the predetermined warning sounds. If the processing a determines that the electrical signals comprise one or more of the predetermined warning sounds, the processing unit is adapted to transmit a warning to one or more of the speakers and the vibration generators.

In another aspect of the invention, the speakers, upon receipt of the warning, output an auditory signal to the user.

In still another aspect of the invention, the vibration generators, upon receipt of the warning, generate vibratory feedback.

In still a further aspect of the invention, the processing unit is further adapted to use machine language techniques to transmit the warning to one or more of the speakers and the vibration generators when the first parameters sufficiently correspond to the second parameters.

In yet another aspect of the invention, the apparatus further comprises one or more motion sensors located on one or more of the pair of earpads or smart earplug companion device, with the motion sensors transmitting information to the processing unit regarding detected movement of the earpad, and with the processing unit further adapted to transmit the warning if the detected movement shows no change for a predetermined amount of time. This prevents unnecessary notifications which may cause user start ignoring similar alarms.

In still yet another aspect of the invention, the apparatus further comprises one or more proximity sensors located on one or more of the pair of earpads, with the proximity sensors transmitting information to the processing unit regarding changes in distance between the proximity sensors and an object, and with the processing unit further adapted to transmit the warning if the distance between the proximity sensors and the object is decreasing. Proximity sensors can be located on the hard hat, earplugs, earmuff or the companion device. Signals received from proximity sensor can tell if the moving object (detected by backup alert detection for example) is approaching the user or getting farther from user. Is it coming from blind spots of the user or in front of the user in which user may have visual of the object? Based on the location and direction of the movement of the object we decide whether or not we notify the user of hazard of being hit by a vehicle.

In still a further aspect of the invention, the apparatus further comprises a transceiver for transmitting the warning to an external device.

In another aspect of the invention, an apparatus for hearing protection for a user comprises a pair of earbuds, a control unit, one or more microphones, one or more speakers, one or more vibration generators, a processing unit, and a notification module. Each of the earbuds is adapted to be placed over an ear of the user. The control unit is connected to each of the pair of earbuds. The microphones are adapted to convert acoustic signals into electrical signals, with the microphones located one or both of the earbuds and the control unit. The speakers are located on each of the pair of earbuds, with the speakers adapted to direct sound towards the ear. The vibration generators are located on one or both of the earbuds and the control unit, with the vibration generators adapted to generate vibratory feedback to the user. The processing unit is in the control unit, with the processing unit adapted to compare first parameters of the electrical signals from the microphones with second parameters of predetermined warning sounds to determine whether the electrical signals comprise one or more of the predetermined warning sounds, and if the processing unit determines that the electrical signals comprise one or more of the predetermined warning sounds, the processing unit is adapted to generate a warning. The notification module is connected to the speakers and the vibration generators and adapted to receive the warning from the processing unit and to transmit the warning to the speakers and the vibration generators.

In still another aspect of the invention, the control unit further comprises a display configured to display messages to the user.

In still yet another aspect of the invention, the apparatus further comprises an external device separate from the earbuds and the control unit. The external device comprises an external processor and one or more external sensors. The external processor is in communication with the processing unit and the notification module, and the external sensors are in communication with the external processor. The processing unit is adapted to transmit the warning to the external processor, with the external processor, based on information from the external sensors, able to transmit a message to the notification module to prevent transmission of the warning to the speakers and the vibration generators.

In still a further aspect of the invention, the external sensors comprise location sensors to detect a location of the external device.

In another aspect of the invention, a safety apparatus for a user comprises a hearing protection device and a communications device. The hearing protection device is worn over the ears of the user. The communications device is carried by the user, with the communications device in communications with the hearing protection device. The communications device comprises one or more sensors, one or more microphones, and a processing unit. The sensors comprise one or more of the following: accelerometers, gyroscopes, motion sensors, and heart rate sensors. The microphones convert acoustic signals into electrical signals. The processing unit is adapted to receive information from the one or more sensors and to receive the electrical signals, with the processing unit further adapted to generate a warning to the hearing protection device based, at least in part, on the information from the sensors and from the electrical signals.

In still another aspect of the invention, the communications device further comprises a transceiver for communicating with another one of the communications devices.

In still yet another aspect of the invention, the communications device is adapted to transmit the warning to the another one of the communications devices through the transceiver.

In a further aspect of the invention, the processing unit is further adapted to compare first parameters of the electrical signals from the microphones with second parameters of predetermined warning sounds to determine whether the electrical signals comprise one or more of the predetermined warning sounds, and if the processing unit determines that the electrical signals comprise one or more of the predetermined warning sounds, the processing unit is adapted to generate the warning.

In a still further aspect of the invention, the communications device is a wristband.

In a still yet further aspect of the invention, the communications device comprises a user interface, with the user interface adapted to accept haptic input from the user.

The hearing protection device may also contain sensors, including but not limited to accelerometers, gyroscopes, or compasses (magnetometers) and use the inputs from these devices to further assess the health and safety situation of the user and take the appropriate action as needed. The readings from the sensor can also act as input methods by the user for more complex interactions with the device.

In another embodiment of the invention, a safety apparatus for a user comprises a mobile device and a tag. The mobile device is configured to play audio to the user. The tag comprises a clip configured to attach to the user, one or more microphones, a tag processor, and a tag transceiver. The microphones are configured to convert acoustic signals into electrical signals. The tag processor is configured to process the electrical signals and to detect if the electrical signals correspond to one or more predetermined warning sounds. The tag transceiver is configured to communicate wirelessly with the mobile device. The tag transceiver is further configured to transmit an alert to the mobile device when the tag processor detects that the electrical signals correspond to one or more of the predetermined warning sounds. The mobile device is configured to stop playing of the audio upon receipt of the alert from the tag transceiver.

In still another embodiment of the invention, the tag further comprises one or more tag vibrators. The one or more tag vibrators are configured to cause vibration when the tag processor detects that the electrical signals correspond to one or more of the predetermined warning sounds.

In yet another embodiment of the invention, the tag further comprises a tag display configured to display information to the user.

In still yet another embodiment of the invention, the tag transceiver is further configured to receive information from the mobile device regarding the audio played by the mobile device. The tag display is further configured to display the information.

In one embodiment, a safety system comprises a plurality of communications devices. Each of the communications devices is configured to be in communication with one or more of other ones of the communications devices. Each of the communications devices comprises a transceiver. Each of the communications devices is adapted to determine an approximate distance to the one or more of the other ones of the communications devices and to generate a warning when one of the approximate distances is less than a predetermined distance. A determination of the approximate distance to the one or more of the other ones of the communications devices is based, at least in part, on one or both of received signal strength indicator (RSSI) or global positioning system (GPS), depending on the predetermined distance.

In another embodiment, the communications device is further adapted to use machine learning techniques and RSSI to determine the approximate distance.

In yet another embodiment, one or more of the communications devices are carried by users and one or more of the communications devices are configured to be mounted in vehicles.

In still yet another embodiment, the vehicles comprise a display configured to display a relative location of other ones of the communications devices.

In a further embodiment, the safety system further comprises a server. The server is configured to communicate with one or more of the communications devices and to receive from the one or more of the communications devices a respective location of each of the one or more of the communications devices.

In still a further embodiment, the safety system further comprises a mobile device and a code. The code is adapted to be scanned by the mobile device. Upon scanning of the code by the mobile device, the mobile device is configured to transmit to the server a location of the mobile device. The server is configured to generate the warning when the mobile device is less than the predetermined distance to one of the communications devices.

In still yet a further embodiment, the safety system further comprises a mobile device and a code. The code is adapted to be scanned by the mobile device. Upon scanning of the code by the mobile device, the mobile device is configured to determine, based, at least in part, on the power of signals received from the mobile device from one or more of the communications devices, an approximate distance to the one or more of the communications devices and to generate a warning when one of the approximate distances is less than a predetermined distance.

In another embodiment, a communications device for use in a vehicle with a plurality of speakers comprises a plurality of microphones and a processing unit. The plurality of microphones are adapted to convert acoustic signals into electric signals, with the plurality of microphones are located on various locations on the vehicle. The processing unit is coupled to the microphones, and the processing unit is configured to receive the electrical signals from the plurality of microphones. The processing unit is further configured to compare first parameters of the electrical signals received from the microphones with second parameters of predetermined sounds of interest to determine whether the electrical signals comprise one or more of the predetermined sounds of interest and a direction from which the acoustic signals are originating. Upon the processing units determining that the electrical signals comprise one or more of the predetermined sounds of interest, the processing unit is configured to cause one or more of the speakers to play the predetermined sounds of interest, the processing unit selecting the particular ones of the one or more of the speakers to provide directionality to the predetermined sounds of interest.

In still another embodiment, the processing unit is further configured to determine whether a source of the predetermined sounds of interest is moving away from or towards the communications device by comparing differences between relative magnitudes of a fundamental frequency and associated frequency bands of the predetermined sounds of interest over time.

In still yet another embodiment, the predetermined sounds of interest correspond to jet engine sounds.

In another embodiment, a system for communications comprises a server and one or more communications devices. Each of the communications devices comprises audio input and output and a transceiver configured to communicate with the server and with other ones of the communications devices. The communications devices are configured to communicate with other ones of the communications devices either (a) directly, when the communications devices are within a certain distance of each other, or (b) through the server.

In still another embodiment, the communications devices are configured to allow for selection between communicating with other ones of the communications devices directly or through the server.

In a further embodiment, the communications devices are configured to communicate with other ones of the communications devices directly using Bluetooth Low Energy (BLE) when the communications devices are within the certain distance of each other.

In still a further embodiment, the communications devices are configured to communicate with other ones of the communications devices through the server using one or both of cellular networks or Wi-Fi.

In still a further embodiment, the system further comprises one or more radio transmitters in radio communications with each other. The one or more of the radio transmitters are coupled to one or more of the communications devices to relay communications among the radio transmitters and other ones of the communications devices.

In still yet a further embodiment, the one or more radio transmitters and the one or more communications devices are grouped into one or more channels. Communications are limited to the one or more radio transmitters and the one or more communications devices within each of the channels.

In another embodiment, the server comprises an interface, the interface configured to allow for access to communications within different ones of the channels.

In still another embodiment, the server is configured to prioritize certain ones of the connections between two of the communication devices.

In still yet another embodiment, the server is configured to prioritize certain ones of the connections, depending, at least in part, on a charging status of the communications devices, an installation location of the communications devices, and a current status of the communications between the communication devices.

The foregoing was intended as a broad summary only and of only some of the aspects of the invention. Other aspects of the invention will be more fully appreciated by reference to the detailed description and the drawings.

### BRIEF DESCRIPTION OF THE DRAWINGS

The invention will be described by reference to the detailed description and to the drawings thereof in which:

FIG. 1 shows the system in accordance with the invention;

FIG. 2 shows one embodiment of the hearing protection device of the present invention;

FIG. 3 shows another embodiment of the hearing protection device of the present invention;

FIG. 4 shows another embodiment of the system, comprising a wristband;

FIG. 5 shows another embodiment of the system, comprising an article;

FIG. 6 shows a process flow for the detection of warning sounds in accordance with one embodiment of the invention;

FIG. 7 shows a block diagram of the components in accordance with one embodiment of the invention;

FIG. 8 shows another embodiment of the system, comprising a tag; and

FIG. 9 illustrates a computing system that may be used to implement various aspects of the embodiment;

FIG. 10 illustrates another embodiment;

FIG. 11 is a block diagram of the components of the communications device;

FIG. 12 illustrates yet another embodiment;

FIG. 13 illustrates one possible of the communications device microphones on a vehicle;

FIG. 14A depicts one possible arrangement of the communications device microphones;

FIG. 14B shows a process flow for detecting a sound of interest;

FIG. 15 is a table depicting one possible management of communication priorities; and

FIG. 16 illustrates another embodiment.

### DETAILED DESCRIPTION OF THE INVENTION

In the following description, various aspects of the illustrative implementations will be described using terms commonly employed by those skilled in the art to convey the substance of their work to others skilled in the art. However, it will be apparent to those skilled in the art that embodiments of the present disclosure may be practiced with only some of the described aspects. For purposes of explanation, specific numbers, materials and configurations are set forth in order to provide a thorough understanding of the illustrative implementations. However, it will be apparent to one skilled in the art that embodiments of the present disclosure may be practiced without the specific details. In other instances, well-known features are omitted or simplified in order not to obscure the illustrative implementations.

In the following detailed description, reference is made to the accompanying drawings that form a part hereof, wherein like numerals designate like parts throughout, and in which is shown by way of illustration embodiments in which the subject matter of the present disclosure may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

In some cases, various operations will be described as multiple discrete operations in turn, in a manner that is most helpful in understanding the present disclosure; however, the order of description should not be construed to imply that these operations are necessarily order dependent. In particular, these operations need not be performed in the order of presentation.

For the purposes of the present disclosure, the phrase “A and/or B” means (A), (B), (A) or (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

The term “coupled with,” along with its derivatives, may be used herein. “Coupled” may mean one or more of the following. “Coupled” may mean that two or more elements are in direct physical or electrical contact. However, “coupled” may also mean that two or more elements indirectly contact each other, but yet still cooperate or interact with each other, and may mean that one or more other elements are coupled or connected between the elements that are said to be coupled with each other. Furthermore, it is to be understood that the various embodiments shown in the Figures (“FIGs.”) are illustrative representations and are not necessarily drawn to scale.

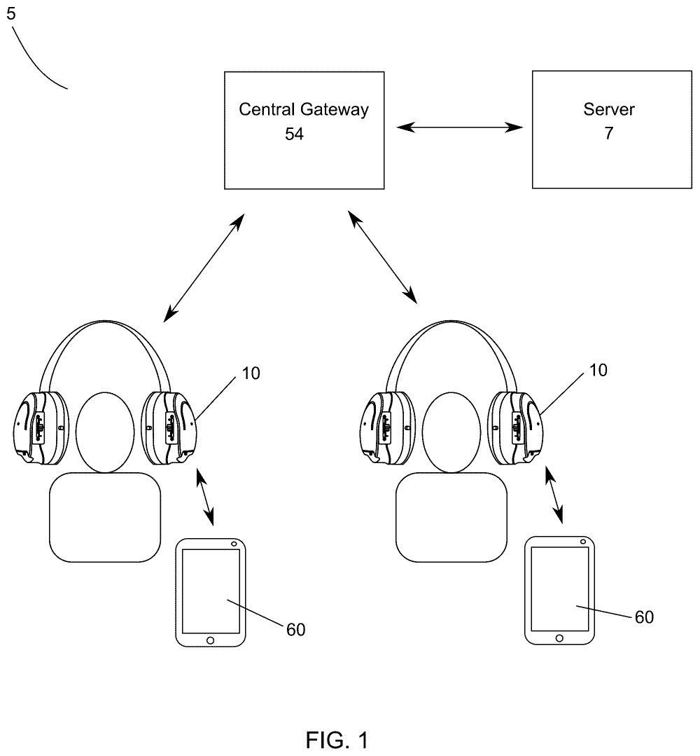

Referring to FIGS. 1 to 9, the present invention comprises a system 5 with one or more hearing protection devices (HPD) 10. The HPD 10 may take any number of forms, including that of a pair of headphones. The HPD 10 may also incorporate one or both of passive and active noise reduction techniques.

In addition, the HPD 10 comprises a processor 12 that is configured to identify certain predetermined warning sounds (e.g. sirens, horns, backup alerts, designated spoken phrases, etc.).

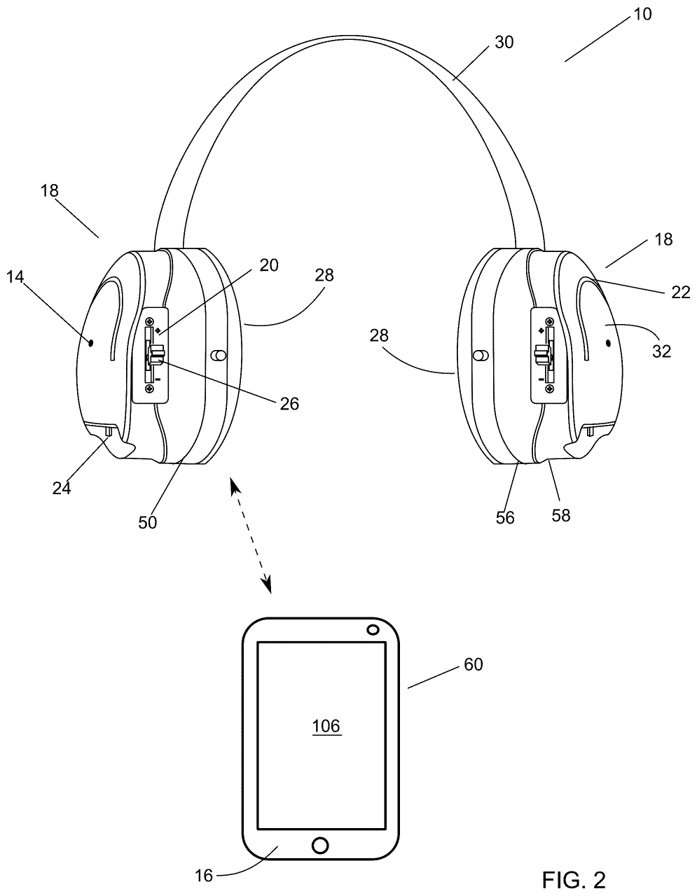

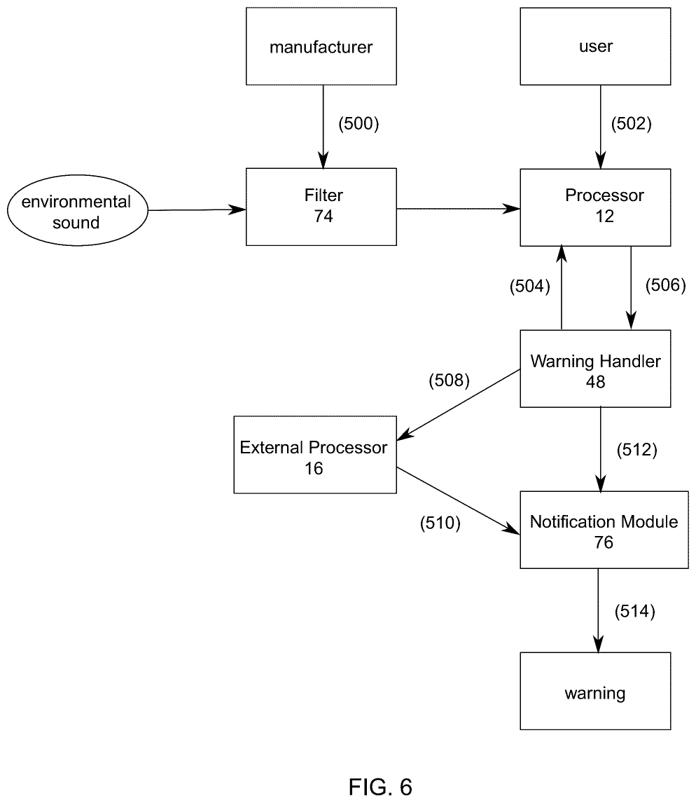

The HPD 10 comprises one or more arrays of acoustic signal to electric signal converters, such as microphones 14 (of FIG. 2), for detecting and capturing sound external to the HPD 10. The microphones 14 are configured to convert the detected sound into electrical representations of the sound. These electrical representations are then sent to the processor 12 for processing (e.g. as shown in the flowchart of FIG. 6) in order to identify if the predetermined warning sounds are present in the detected sound. The electrical representations may also be sent to an external processor 16 that is external to the HPD 10 for processing. This may be done through a wired or wireless connection between the HPD 10 and the external processor 16.

Referring to FIG. 2, in one embodiment, the HPD 10 may take the physical form of a conventional earmuff. In this embodiment, the HPD 10 comprises two earpads 18, with each earpad 18 placed on or over an ear of the user. In the embodiment shown, a band 30 may extend between the two earpads 18 and is adapted to fit about the head of the user and to hold the earpads 18 in place over the ears. The HPD 10 may also comprise one or more manual inputs 20 for controlling or adjusting input to the HPD 10. For example, the manual inputs 20 may include a switch 24 to turn on or turn off operation of the HPD 10. The manual inputs 20 may also include a variable input 26, such as a slider or leveler, that allows the user to adjust the degree of sensitivity or volume of the HPD 10. The variable input 26 may be used to adjust the degree of hearing protection for the user. In one embodiment, one or both of the manual inputs and the variable inputs 26 may be located on one or both of the earpads 18. One or more of the microphones 14 may be located on one or both of the earpads 18.

The HPD 10 may also comprise a notification area 22. The notification area 22 may include lights that illuminate to provide visual information. In another embodiment, the notification area 22 may also include a display screen. In the embodiment shown in FIG. 2, the manual inputs 20 and the notification area 22 are located on an exterior surface 32 of one or both of the earpads 18 such that they can be accessed or viewed externally.

One or more of the microphones 14 may be situated at various locations on the HPD 10. The microphones 14 are preferably situated on an exterior surface 32 to allow for better sound detection, such as, for example, on the exterior surface 32 proximate to or on the earpads 18.

The HPD 10 may also comprise one or more speakers 28. In some embodiments, the speakers 28 are embedded within each of the two earpads 18, with any sound generated by the speakers 28 being directed towards the ear of the user. The HPD 10 may also comprise one or more vibration generators 50 that are configured to generate vibratory or haptic feedback to the user. The vibration generators 50 may be located on one or both of the earpads 18 such that any vibratory or haptic feedback may be felt by the user.

In addition, the HPD 10 may comprise one or more ports 38 located on one or both of the earpads 18. The ports 38 may include a Universal Serial Bus (USB) port 40 that may be used for transferring data between the HPD 10 and another external device. Furthermore, the HPD 10 may comprise a battery 42 for supplying electrical power to the various components of the HPD 10. The ports 38, including the USB port 40 may be used to charge the battery 42 from an external power source.

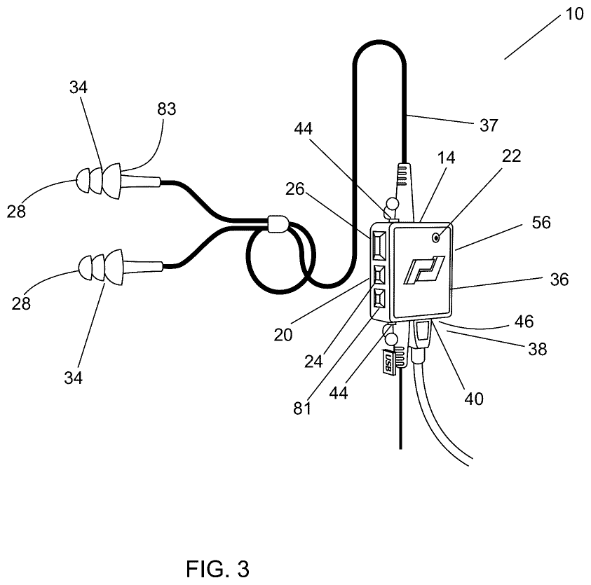

Referring to FIG. 3, in another embodiment, the HPD 10 may take the physical form of conventional earphones. In this embodiment, the HPD 10 comprise two earbuds 34, with the earbuds 34 being electrically connected to a control unit 36 through wire 37. In embodiments, the earbuds 34 may have a high noise rejection rating. The control unit 36 may comprise one or more of the manual inputs 20 (i.e. the switch 24 and the variable input 26). The control unit 36 may also comprise the notification area 22 to provide visual information. The manual inputs 20 may be configured to perform various tasks when activated. In addition, the manual inputs 20 may be configured to perform various tasks when activated in a particular sequence (e.g. when activated twice in quick succession).

In the embodiment shown, one or more of the microphones 14 may be situated on the control unit 36. One or more of the speakers 28 may also be embedded within one or both of the earbuds 34 such that any sound generated by the speakers 28 may be directed to the ear of the user. In one embodiment, one or more of the microphones 14 may also be located on one or both of the earbuds 34.

The control unit 36 may also comprise one or more of the ports 38 for interfacing the HPD 10 with other devices. For example, the ports 38 may include the Universal Serial Bus (USB) port 40 for transferring data between the control unit 36 and an external device. The control unit 36 may comprise the battery 42 within the control unit 36 for supplying electrical power to the various components of the HPD 10, in which case the USB port 40 may also be used to charge the battery 42 using an external power source. The ports 38 may also include an input port 44 for receiving data from other radio devices and may also include an output port 46 that provides a connection for the wire 37 from the control unit 36 to the earbuds 34 (and allows for the transfer of data between the two).

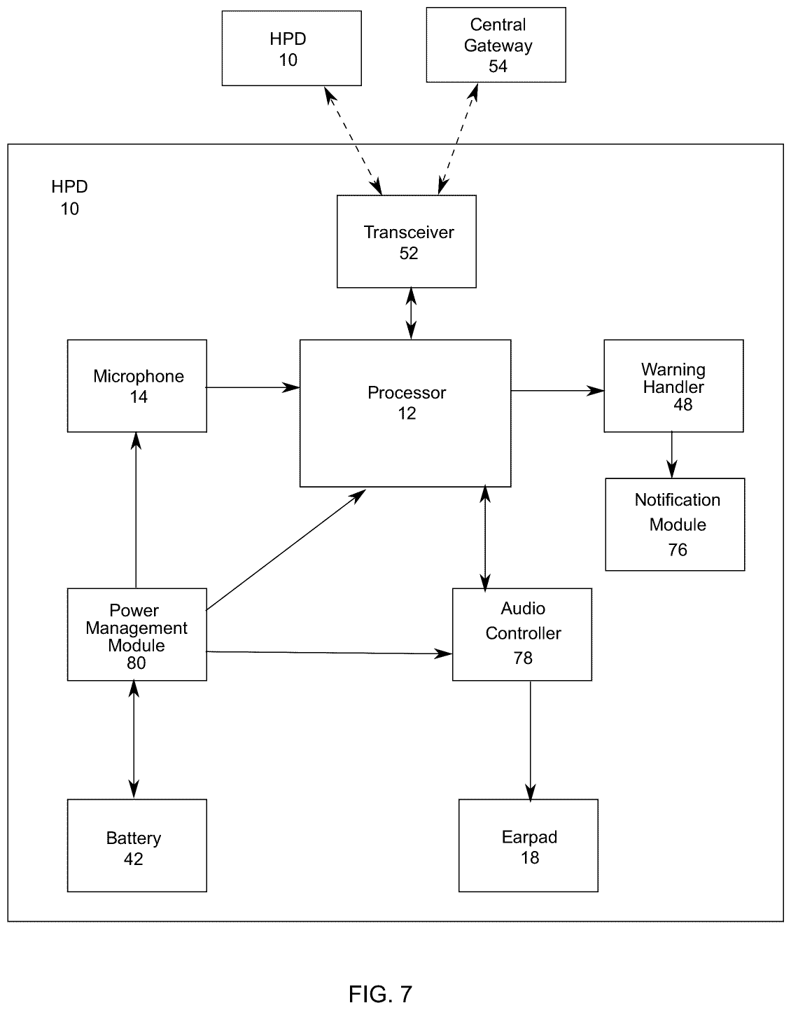

Detection of warning sounds using the HPD 10 will now be described. The detection of warning sounds may be done using supervised machine learning. In particular, the processor 12 (as shown and discussed with respect to FIGS. 6 and 7) may be trained using supervised machine learning techniques. In one embodiment, prerecorded audio samples labelled as being appropriate warning signals are inputted to the processor 12, and through the training process, the free parameters of the detection system will be adjusted by the processor 12. The learning can be transferred dynamically by the user in situ to the processor 12. For example, upon the presence of a new improved warning detection engine, the new values for the free/tunable parameters can be transferred to the processor 12, and the detection engine on the processor 12 can be updated.

The user can also invoke the learning system on the processor 12 when a new warning signal is playing and the processor 12 can extract the signatures of the new warning signal and detect it automatically afterwards.

Moreover, the warning detection system of the processor 12 can also be trained through unsupervised machine learning techniques. For this, a large number of different types of warning sounds are continuously introduced to the processor 12 and over time, the processor 12 is able to distinguish and identify similar warning sounds. In one embodiment, clustering algorithms may be used, such as K-mean clustering to the N-dimensional scattered plot data with each feature on its own axis. The data with similar features can will be clustered together (unsupervised grouping). The cluster information will be used as a method to categorize different warning signals. These unsupervised learning techniques can also be done on the external processor 16, which can then transmit the warning signatures of different types to the processor 12. In embodiments, the external processor 12 is included in a mobile device or remote server communicatively coupled to HPD 10. The grouping/clustering information can then be transferred to the device itself for the future appropriate grouping of the new incoming warning signals.

Moreover, in one embodiment, machine learning techniques or other detecting algorithms may be used to detect characteristics of a particular sound. For example, machine learning techniques may be used to detect the characteristics of a conventional siren. The processor 12 can then remove those characteristics in the frequency domain from a streaming signal. In other words, the processor 12 may be configured to scan the signals (sounds) received from the microphones 14 and if the characteristics of the siren are detected, the processor 12 may be configured to remove those characteristics from signals. In this manner, users (such as fire fighters) can continue to stream signals from the microphones 14 and have those signals played through the speakers 28 except for the sound of the siren. This would allow the user to hear other sounds that may be masked by the siren (which can be very loud).

The HPD 10 can also be configured to detect different type of external sounds and based on their characteristics, interrupt an acoustic stream being provided to a user by turning off the acoustic stream to allow the user to hear the surrounding sounds and/or provide a warning sound. For example, in some embodiments, the HPD 10 detects bicycle rings and based on the speed of approaching and proximity (extracted from the growth rate of the amplitude of the bicycle ring sound), may generate different levels of warning to the user. For example, if the user is listening to music at the time, the HPD 10 may turn off the music and stream the surrounding sounds (e.g. the bicycle rings) to the user. That way, if there is a bicycle rider sounding the bicycle rings, and there is a user in front listening to the music, the HPD 10 can be configured to allow the user to hear the sound of the bicycle ring. Alternatively, the HPD 10 may be configured to turn off the user's music if the HPD 10 recognizes (or detects) the sound of a moving bicycle chain coming closer and closer to the user. Depending of the speed of the bicycle approaching, the user may receive different notifications (e.g. a fast and loud “Beep Beep” sound, a vocal message, a low-frequency “Beep Beep” sound, etc.). The HPD 10 may also vibrate and illuminate to notify the user. Alternatively, the HPD 10 may cause an audible message (such as “Watch out!”, “Bike!”, or “Bike coming!” in English or different languages to be played in order to notify the user. In another application it can detect any speech and turn off the music and turn on the surrounding sounds.

In addition, the HPD 10 may be configured to not only detect bicycle rings to determine if a bicycle is approaching but may detect other sounds that may be associated with an approaching bicycle. For example, the HPD 10 may be configured to detect the sound the chain of the bicycle makes when moving. The HPD 10 may be configured to determine, based on the sound made by the chain, whether the bicycle is moving towards or away from the user. Furthermore, the HPD 10 may be configured to detect words that may be spoken by individuals on the bicycle or nearby. For example, the HPD 10 may be configured to detect if someone says “Watch out!”, “Bike!”, “Bike coming!”, or the like, in English or different languages.

The HPD 10 comprises a warning handler 48 that is triggered when a warning sound (e.g. a siren, a bicycle ring, etc.) is detected. The warning handler 48 may be a separate unit from the processor 12, or alternatively, it may be integrated with the processor 12. Thus, in embodiments, warning handler 48 includes a separate hardware circuitry unit from the processor 12, software run on or residing in processor 12 or combination thereof. In the event that the processor 12 detects one or more of the warning sounds, the processor 12 may trigger the warning handler 48 in order to send a notification to the user. The notification may take the form of a vibratory signal (i.e. haptic feedback), an auditory signal (e.g. played to the speaker 28), or a visual signal (e.g. the illumination of lights or the display of a text message through the notification area 22), or in the form of electromagnetic signals (e.g. radio, cellular, Wi-Fi, Bluetooth signals, etc.) to an external handheld device that the user or others are using. For example, in one embodiment, the detection of one or more of the warning sounds by the processor 12 may result in the processor 12 triggering the warning handler 48 to cause one or more of the vibration generators 50 to trigger, creating a vibration in the HPD 10 that may be felt by the user. The HPD 10 may be configured to provide vibrations at different locations on the HPD 10, depending on the location of the vibration generators 50 on the HPD 10. In addition, the vibrations may be customizable in terms of intensity and/or pattern of vibration.

In one embodiment, the HPD 10 may comprise a notification module 76 that is configured to accept notifications from the warning handler 48 and effects notification to the user. The notification module 76 is preferably connected to one or more of the components responsible for alerting the user, such as the speakers 28, the notification area 22, etc. The notification module 76 may be a separate unit from the processor 12 and/or the warning handler 48. Alternatively, the notification module 76 may be integrated with one or both of the processor 12 and the warning handler 48.

Furthermore, in one embodiment, the HPD 10 may also comprise a transceiver 52 connected to the notification module 76 and is configured to transmit the notification wirelessly to other nearby users that are also wearing a similar one of the HPD 10 (e.g. 10a) or to a central gateway 54, that is part of the system 5. The central gateway 54 may be a computer that is configured to receive and/or relay notifications. These wireless communications may be carried out using Wi-Fi, cellular, Bluetooth, or the like.

In addition to being triggered by the detection of a predetermined warning sound, the notification may also be triggered by other means, including:
(a) The receipt of a message and/or signal from an external software application (e.g. an app installed on a mobile device), an emergency broadcasting system, other ones of the HPD 10, or other external devices (such as a wristband, as described later). In one embodiment, one of the HPD 10 may act as a “broadcaster” of messages and/or signals to other ones of the HPD 10. For example, if one of the HPD 10 receives a message and/or signal, that particular one of the HPD may broadcast or transmit (using the transceiver 52) the same message and/or signal to other ones of the HPD 10, acting as a repeater so the other ones of the HPD 10 is able to receive the message and/or signal. This transmission can happen in a peer-to-peer mode to ensure the other ones of the HPD 10 receives (using the transceiver 52) the message and/or signal even if they are not in the range to receive the original message and/or signal. In this embodiment, the other ones of the HPD 10 may have cause to have a notification triggered, which would then be handled by the notification module 76 as described above.
(b) The detection by the HPD 10 of certain words in one or more languages (e.g. “Watch out!” or the like).
(c) The receipt a text message or a phone call (e.g. such as through the app). In this embodiment, the HPD 10 may be configured to accept a notification from an external mobile device (such as a cellular phone) that a text message or a phone call has been received. The notification module 76 may be triggered to provide notification to the user of the text message or the phone call.

In another embodiment, the HPD 10 may also comprise one or more motion sensors 56. The motion sensors 56 are configured to record and transmit data to the processor 12 regarding the movement of the HPD 10. If the processor 12 determines, based on the data received from the motion sensors 56, that the HPD 10 is falling, the processor 12 may determine that the user (who is wearing the HPD 10) is likely falling and may also trigger the warning handler 48. Alternatively, if the processor 12 determines, based on the data received from the motion sensors 56, that the HPD 10 has not moved for a certain period of time, the processor 12 may also trigger the warning handler 48 (as this may be an indication that the user may be in distress and unable to move). In either scenario, the warning handler 48 may cause a notification to be generated to the notification module 76. The notification 76 may cause the user to be alerted (e.g. through the vibration generators 50, the notification area 22, the speakers 28, etc.). In addition, the notification module 76 may cause the transceiver 52 to generate a message and/or signal to the central gateway 54 or to other ones of the HPD 10. Furthermore, the processor 12 may cause the transceiver 52 to transmit a telephone call, an email, or some other alert to others. In this manner, the HPD 10 may be configured to generate an alert (acoustic or electromagnetic).

In another embodiment, the system 5 may comprise one or more proximity sensors 58 to record and transmit data to the processor 12 regarding changes in distance between the proximity sensors 58 and other objects. The proximity sensors 58 may include optical proximity sensors, capacitive proximity sensors, ultrasonic proximity sensors, or other types of suitable proximity sensors. In embodiments, the information may in turn cause the warning handler 48 to generate an appropriate notification. The warning handler 48 may be hardware or software based. For example, if a truck is moving towards the HPD 10, the proximity sensors 58 would be able to detect the changes in distance between the truck and the HPD 10. If the processor 12 determines, based on the data received from the proximity sensors 58, that an object is approaching the user, the processor 12 may be configured to trigger the warning handler 48. This determination by the processor 12 depends, at least in part, on the rate that the object is approaching the user, the estimated size of the object, and the direction from which the object is approaching the user. This information may be provided to the processor 12 by the proximity sensors 58. The processor 12 may also take into account or other information, such as any conventional backup alert or siren sounds detected by the microphones 14. Using the information from the proximity sensors 58 and/or the microphones 14, the processor 12 may also be configured to determine the nature of approaching object (e.g. if the processor determines that the backup alert corresponds to conventional backup alerts used by trucks) and cause the warning handler 48 to generate the appropriate notification accordingly. This technique can be in particular important since it can detect in all directions from the HPD 10 and therefore can detect objects that are approaching the HPD 10 from the blind sides of the user, thereby improving the awareness of the user.

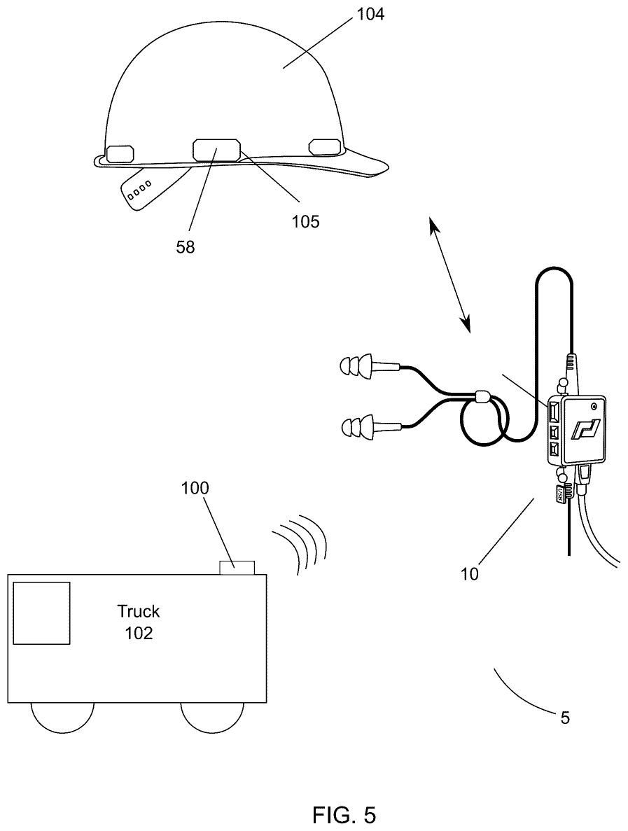

In yet another implementation, referring to FIG. 5, the system 5 further comprises one or more radio frequency (RF) broadcaster 100 that may be provided and installed in series with, e.g., a conventional backup alert system of trucks 102 or other vehicles with backup alert. The RF broadcaster 100 is configured to transmit a designated pattern of data (such as status of the vehicle, geographical location of the vehicle, etc.) with a designated RF power only when the truck 102 or vehicle is backing up, and when the backup alert is activated. The transceiver 52 on the HPD 10 may be configured to periodically scan for such RF signals and, based on the designated pattern, the processor 12 may be configured to recognize it as a backup alert and based on the received RF power, to determine the approximate distance of the truck 102 (or vehicle) that is backing up. For example, if the processor 12 determines that the signal from the RF broadcaster 100 is getting stronger, the processor 12 may determine that the truck 102 is getting closer (physically) (also observable from mapped real time distance) to the HPD 10, and thus the user. This may result in the warning handler 48 being triggered by the processor 12. Alternatively, if the processor 12 determines that the signal is getting weaker, the processor 12 may determine that the truck 102 is moving farther away. This is in particular useful if there are multiple ones of the trucks 102 present, with more than one backup alert operating at the same time. In these circumstances, it may not be possible to rely solely on acoustic detection, as it losses its accuracy.

In another embodiment, the system 5 may also comprise one or more articles 104 that may be worn by the user. The articles 104 may include, but are not limited to, hard hats, vests, wristbands, etc. A plurality of article microphones 105 may be placed on the articles 104. The data received by the article microphones 105 may be transmitted to the HPD 10 for further processing. For example, the article microphones 105 may transmit data to the HPD 10 using Bluetooth. Alternatively, the article microphones 105 may transmit data between themselves using Bluetooth, before the data is transmitted by one of the article microphones 105 to the HPD 10. The data from the article microphones 105 may be used by the processor 12 to determine the direction of oncoming objects by triangulating data from the plurality of article microphones 105.

The motion sensors 56 and the proximity sensors 58 are preferably located on the HPD 10 (such as on the control unit 36, the earbuds 34, or the earpads 18). For example, where the HPD 10 is in the form of earmuffs, the motion sensors 56 and/or the proximity sensors 58 may be located on or proximate to one or both of the earpads 18. Alternatively, the motion sensors 56 and the proximity sensors 58 may be located externally from the HPD 10 and transmit data (wired or wirelessly) to the processor 12. In another implementation, where the HPD 10 takes the form of earphones, the proximity sensors 58 can be located on the control unit 36.

Referring to FIG. 5, in another embodiment, the proximity sensors 58 may be located external to the HPD 10, such as on articles 104. The proximity sensors 58 are able to transmit data to the HPD 10. In various embodiments, the proximity sensors 58 can also be implemented on clothes and vehicles as well.

Furthermore, the processor 12 may be configured to customize the notification in order to provide the user with some information about the nature of the notification. For example, if the notification is in the form of haptic feedback by the vibration generators 50, the vibration patterns may be different for different types of notifications. In one embodiment, the vibrations may be similar to Morse code. In yet another embodiment, where the notification is an auditory signal (e.g. causing the speakers 28 to generate a beeping sound), the beep sounds may be closer together when the object is getting closer to the HPD 10.

Referring to FIG. 5, the processor 12 is configured to detect external sounds in order to identify particular warning sounds and/or spoken words in noisy environments (as received by the microphones 14) and to provide a notification to the user using the warning handler 48. The processor 12 may trigger the warning handler 48 to send the notification to the user in any combination of cases where the source of the warning sound is approaching or moving away from the HPD 10, the source of the warning sound is detected to be nearer than a predetermined threshold, and/or if the HPD 10 is determined to be not moving away from the source of warning signal.

The general direction of warning sounds can be triangulated by comparing the intensity measured at the different ones of the microphones 14 and/or through using data from other ones of the HPD 10 received by the transceiver 52 via wireless communication.

In another embodiment, the processor 12 may be configured to trigger the warning handler 48 to generate the notification to the user if the processor 12 determines that the warning sound or signal originates from a certain direction with respect to the user. For example, the processor 12 may be configured to trigger the warning handler 48 if the processor 12 determines that the warning sound is coming from the user's blind spots (i.e. beyond the user's normal field of vision). For example, where the HPD 10 is in the form of an earmuff, the processor 12 may be configured to approximate the direction from where the warning sound is coming based on the orientation of the earpads 18 (left versus right) and the location of the microphones 14.

For example, the present invention may be used to detect the presence of a backup alert (i.e. a type of warning sound) generated by the truck 102 that is moving backwards. The microphones 14 located on the HPD 10 are configured to pick up external sounds and convert them into electrical signals for processing by the processor 12. The processor 12 processes the electrical signals and looks for certain electrical signatures (e.g. a signature corresponding to the backup alert) in time and/or frequency domains. These signatures may include (but are not limited to) the frequencies of interest, variance of frequencies of interest, variance of amplitude of frequencies of interest, the duration of the warning sounds, the period of the repeating warning sounds, and physical direction of the warning sounds.

The processor 12 is also configured to be able to detect whether the source of the sounds (e.g. the truck 102) is approaching the HPD 10 (and thus the user) or moving away from the HPD 10 (and thus away from the user). For example, the processor 12 may be configured to only trigger the warning handler 48 if the truck 102 is approaching the HPD 10. This can be determined by the processor 12 using methods including, but not limited to, analyzing the associated Doppler effect. The processor 12 may also be further configured to provide the warning handler 48 with information regarding the direction of movement of the object and/or the source of the warning sound (e.g. the backup alert). The warning handler 48 is able to provide the notification module 76 with the information in an understandable form. For example, the notification module 76 may cause the speakers 28 to output, “Truck approaching from behind” or “Backup Alert Detected!”.

Such vocal playback need not be stored on the HPD 10. In one embodiment, the system 5 may also comprise a mobile device 60. For example, the external processor 16 may be located on the mobile device 60. The processor 12 may be configured to detect the warning sound, triggering the warning handler 48 and causing the notification module 76 to transmit a warning signal to the mobile device 60 through the transceiver 52. The mobile device 60 may include a smartphone, a tablet, or the like that is configured by an application 106. The warning signal may, for example, be in the form of a serialized data structure containing the different parameters that specify the exact nature of the warning signal. For example, one field of the data structure might enumerate the type of warning signal (e.g. backup truck versus fire siren), while another field of the data structure might contain the direction that the sound is coming from. A third example field of the data structure may contain information on whether the source of the sound is physically approaching the user.

Once the mobile device 60 receives the data structure, the application 106 can take the appropriate action in response to the data structure. For example, the mobile device 60 may be configured by the application 106 to generate an appropriate voice warning via a text-to-speech mechanism, open a voice stream channel to the HPD 10 (through the transceiver 52) and stream the vocal warning to the HPD 10 for playback through the speakers 28. Additionally, the mobile device 60 may also cause other forms of feedback to the user, such as vibrations. The vibrations may be in a certain pattern, depending on the nature of the warning signal.

The mobile device 60 can also be used (through the application 106) to adjust the settings for the HPD 10, such as configuring an identifier (e.g. name) for the HPD 10 and changing the sensitivity of the HPD 10 (e.g. configuring the approximate distance of the warning sound at which the processor 12 will trigger the warning handler 48). The mobile device 60 can also identify the user through the name and media access control (MAC) address of the mobile device 60 and using the location of mobile device 60 (such as through Global Positioning System), estimate the location of the user.

This, in particular, may be used in case of emergencies to determine whether certain users are still located in particular areas, depending on the locations of users as determined by the HPD 10 and the mobile devices 60.

In another embodiment, the HPD 10 may be configured to provide a streaming functionality. For example, the microphones 14 on the HPD 10 may be configured to detect and record the surrounding sounds, and the processor 12 can be further configured to play, in real-time, the detected and recorded audio sounds through the speakers 28 after applying denoising and warning detection algorithms on them. In this manner, the user can listen to the surrounding environment without hearing certain noises, increasing awareness of his or her surroundings.

The processor 12 is able to detect the main frequencies of noise that is considered unwanted and reject them (during streaming) by the use of a number of filters 74. In one embodiment, the number of filters 74 of FIG. 6 may be from 1 to 7. The filters 74 are a combination of one or more of low-pass, high- pass, notch, and/or band-pass filters. The characteristics (such as type, quality factor, or bandwidth) and frequency of each filter 74 may be adjusted by the user or noise cancellation system versus time. The filters 74 are able to dynamically track the main frequencies of noise considered unwanted and attenuate them. For example, the processor 12 may be previously provided with certain patterns or frequencies of noises that are considered to be unwanted or undesirable.

The processor 12 may be configured to reject common unwanted noise by subtracting the sounds collected by different ones of the microphones 14 from each other with designated gains. This would allow the processor 12 to pick the sounds of sources located in a particular direction (e.g. the front of the HPD 10) while subtracting the sounds that are coming from other sources in other directions.

In yet another embodiment, the processor 12 may be configured to reject or attenuate background noise by obtaining the frequency spectrum of the background noise and subtracting that from the stream of the incoming sound. In this manner, the detection of speech and warning sounds will be improved while background noise will be attenuated. One method to achieve noise profile is window averaging the incoming sound in frequency or time domains or weighted combination of time and frequency domains. By adjusting the window size, the processor 12 may be configured to pick up certain noises in the background. This noise profile may later on subtracted from the incoming spectrum of sounds.

In another embodiment, the HPD 10 may be configured to provide adjustable acoustic protection. For example, the processor 12 may be configured to provide different levels of acoustic protection depending on the volume and duration of exposure of the external noise. The processor 12 may adjust the protection to the best suited Noise Reduction Rating (NRR). The processor 12 may also measure the power (or equivalently, sound pressure level) of the external noise and determine whether the user is required to put on the HPD 10.

Noise cancellation and rejection can be done in combination of methods in both time and frequency domains. In one method, the noise rejection can be done by subtracting the noise spectrum from the incoming signals spectrum and therefore improve the vocal spectrum. It can also be done in time domain by rejecting loud and high intensity signals and attenuating the signal to a safe level in a fast method (dynamic compression). In yet another method, noise rejection can be done by the use of combination of different ones of the microphones 14 and subtracting their signals in different (or the same) weight in a way that the signal that carries more vocal sound has higher weight and get subtracted by microphone output signal which has the same or less amount of background noise and/or vocal sound. This method may be referred to as “multiple mic noise cancellation”.

In yet another embodiment, the sounds from two or more of the microphones 14 may be subtracted through a beam-forming algorithm (e.g. adding a dynamic delay to one or more microphone signals prior to subtractions), so that only certain directions receive high signal gain while some directions reject all incoming sounds. This can be improved by dynamically adopting the weight and polarity of the subtraction from two or more of the microphones 14 and in that way generates dynamically programmable “null” angles where sounds from these angles are more attenuated. This can be in particular useful when the user wants to cancel sounds from loud factory instruments that are approaching to user from certain angles. This may be referred to as “noise cancellation with dynamic beam forming”.

In another embodiment, the HPD 10 may identify sirens from firetrucks by extracting the frequency and characteristics of the siren. The processor 12 may be configured to use these characteristics and to remove the siren sound in time or frequency domain using one or combination of noise cancellation methods and algorithms. In this manner, a firefighter that is using the HPD 10 may be able to hear his or her surroundings and the street sounds without hearing the sirens from nearby firetrucks. In one implementation the processor 12 may be configured to find the characteristics of the siren (through machine learning or preloaded data given by the manufacturer) and based on that, remove the component of the siren from frequency or time domain component of the incoming sound. After removal, the sound with attenuated siren sound can be played by the speakers 28 to allow the user to be more aware of his or her surroundings.

In another embodiment, the processor 12 may be configured to measure the volume of the external noise detected by the microphones 14, to measure the duration of its exposure to the user, to measure the time that the user has been exposed to the external noise, and to determine the weighted noise power (in dBA) from the noise profile, and based on a combination of this information, make a determination as to the appropriate level of Noise Protection Rating (NPR). This determination may be made, at least in part, based on recommended standards set by governmental or safety authorities. Based on this determination, the processor 12 may be configured to adjust the level of noise protection for the HPD 10.

The processor 12 may be configured to check that the sound outputted to the user through the speakers 28 remains below a certain threshold (e.g. 85 dB). If a sudden loud noise is detected by the processor 12, the processor 12 may be configured to reduce the amplification gain (the gain on converting the acoustic signal to a digital signal) as soon as possible to ensure that the user will always receive sound at a consistent volume level through the speakers 28.

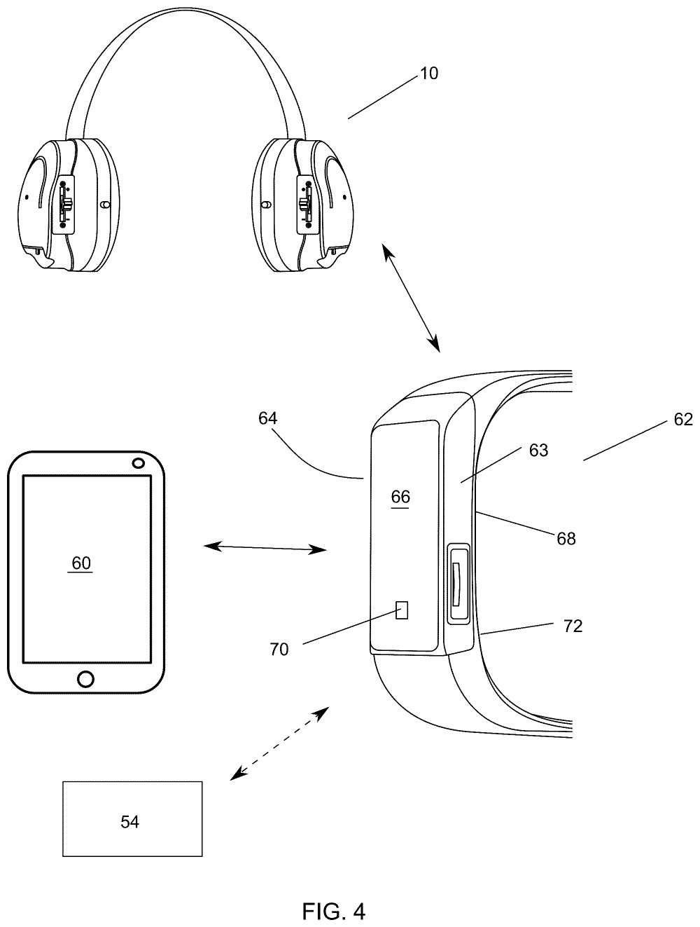

Referring to FIG. 4, the system 5 may also comprise a wristband 62 that is in wireless communications with the HPD 10 through a wristband transceiver 64 located on the wristband 62. The wristband 62 may be worn on the wrist of the user and comprises a wristband interface 66. The wristband interface 66 may comprise a touchscreen (e.g. with a graphical user interface), a button, or the like. The wristband 62 can act as both a mechanism for input to one or more of the HPD 10 and/or one or more of other ones of the wristbands 62 at the same time. The wristband 62 comprises a wristband processor 63 for controlling and receiving input from various components on the wristband 62.

In one embodiment, the wristband processor 63 may be configured to allow the wristband 62 to communicate with the HPD 10 that the user is wearing as well as another one of the HPD that another user is wearing. The wristband 62 can also be paired with the mobile device 60 that the user is carrying, or it may also be paired and communicate with the central gateway 54. As an example, the user may tap on the wristband interface 66 in order to issue commands to the wristband 62. If the user wishes to communicate with another user through the HPD 10, the user may tap on the wristband interface 66. In one example, different commands may be assigned to different tap patterns. For example, communicating with user A may involve tapping on the wristband interface 66 once, while communicating with user B may involve tapping on the wristband interface 66 twice. Similarly, communicating with all users simultaneously may involve tapping on the wristband interface 66 six times. The particular tap patterns may be configured by the user.

In one embodiment, the wristband 62 may be paired with the HPD 10 using RSSI values of the connection protocol (e.g. BLE) used to communicate between the wristband 62 and the HPD 10. For example, the user may use the wristband interface 66 to enter into “pairing mode” and then bring the wristband 62 physically closer to the HPD 10 (e.g. within 10 centimeters) or alternatively, in physical contact with the HPD 10. When the wristband 62 is physically closer to the HPD 10 (or in physical contact with the HPD 10), the HPD 10 will detect a high RSSI value from the wristband 62, which the processor 12 may interpret as an indication that the wristband 62 is to be paired with the HPD 10.

The wristband 62 may also comprise wristband sensors 68, which may include one or more of accelerometers, gyroscopes, motion sensors, GPS, heat sensors, infrared proximity sensors and/or heart rate sensors that provide input to the wristband processor 63. For example, the wristband sensors 68 are able to detect if the wristband 62 has not moved for a certain period of time or if the user is not vertical. Upon receipt of this information by the wristband processor 63, the wristband processor 63 may be configured to cause the information to be transmitted by the wristband transceiver 64 to the central gateway 54 or the HPD 10 for further processing. The wristband 62 may comprise wristband vibration generators 72 for providing haptic feedback to the user, upon receipt of appropriate commands from the wristband processor 63.

The wristband 62 may also comprise wristband microphones 70 for recording sounds and sending the data to the wristband processor 63 for further analysis (e.g. to determine if they correspond to warning sounds). Alternatively, the wristband processor 63 may be configured to simply cause the data to be transmitted to the HPD 10 using the wristband transceiver 64 for processing and determination by the HPD 10 of whether a warning sound is present.

The input signals that are generated by the wristband 62 may not be purely active signals. The user need not interact or “do something” with the wristband 62 in order to generate an input signal. The lack of interaction or a change in a signal pattern may be enough to trigger an action. As an example, if the wristband sensors 68 detect a stoppage or pause in movement, or some other measurable change, the wristband processor 63 may cause a notification to be generated. For example, if the user has fallen asleep, the wristband sensors 68 may detect that the pattern of motion (based on gyroscopes and accelerometers) has changed and that the heart rate (based on the heart rate sensors) has changed. From this information, the wristband processor 63 may be configured to make a determination as to whether the user may have fallen asleep. The wristband processor 63 may cause a signal to be transmitted to the HPD 10 that triggers a haptic feedback on the HPD 10 along with some other notification (e.g. to the mobile device 60 or the central gateway 54).

In one embodiment, the wristband processor 63 may have full signal detection embedded in its own hardware so that when it detects a warning sound, it notifies the user using a visual indicator, such as a flashing light, an icon on the wristband interface 66 or through vibration (e.g. using the wristband vibration generators 72) or haptic or taptic feedback. The wristband 62 can update itself through mobile that is connected to the Internet. It can also have BLE and/or Wi-Fi connectivity.

The HPD 10 may be wirelessly connected to the mobile device 60. For example, two users C, D may each be wearing respective ones of the HPD 10 (HPD 10c, 10d), with each carrying one of the mobile devices 60 (mobile devices 60c, 60d) wirelessly connected to their respective HPD 10 (HPD 10c, 10d, respectively). The mobile devices 60c, 60d may be connected to the HPD 10c, 10d, respectively by wireless communications, such as Bluetooth, Wi-Fi, or cellular connections. In this manner, users C, D may communicate with each other through their respective HPD 10c, 10d over virtually unlimited distances (limited only by the range of the connections between the respective mobile devices 60c, 60d). Once user C has connected the mobile device 60c to the HPD 10c, user C will be able to view, on the mobile device 60c, a list of other users that user C can connect to. If user C chooses to connect with user D (using, for example, a push-to-talk button), a corresponding notification will be sent to the mobile device of user D. Once the connection has been established, voice communications may be made between users C, D through the speakers 28c, 28d and microphones 14c, 14d located on the respective HPD 10c, 10d. In addition, it is also possible for user C to connect with a number of other users in order to communicate with them.

In another embodiment, the HPD 10 may use Bluetooth low energy (BLE) to communicate with each other, with the communications being encrypted. For example, if user C wishes to connect to user D, both users C, D simply need to turn on their respective HPD 10 (HPD 10c, 10d). The HPDs 10 are able to automatically connect to each other, and either user C or user D can start the conversation by pressing an appropriate button (e.g. a push-to-talk button) on the HPD 10. If user C wishes to connect to multiple other users (that are in range), all of the users simply need to turn on their respective HPDs 10. The HPDs 10 are able to automatically connect to each other. In one embodiment, the particular HPD 10 on which the user initiated the conversation first (e.g. the first user to press the push-to-talk button) will be designated as the speaker, and all of the other HPDs 10 will be designated as receivers and can only listen to communications from the particular HPD 10. This may be modified by one of the other users pressing on the push-to-talk button on his or her one of the HPDs 10 in a predetermined manner (e.g. pressing three times in a row). At that time, that HPD 10 will now be designated as the speaker, with the other HPDs 10 being designated as receivers.

In another embodiment, the microphones 14 on the HPD 10 may also be used to detect tap sounds made by the user on the HPD 10. The processor 12 may be configured to identify such tap sounds and carry out commands based on those tap sounds. For example, a particular tap pattern may be used to initiate a call with another predetermined user.

Referring to FIG. 6, which shows a process flow in accordance with an embodiment of the invention, the filters 74 may be preconfigured (500) by the manufacturer so that the processor 12 detects certain warning sounds from the general environmental sounds picked up by the microphones 14 and potential proximity and movement sensors. In addition, the processor 12 may also be configured in situ (502), such as by transferred by user and/or through machine learning. In other words, through machine learning, the processor 12 may be trained to detect further warning sounds that have not been preconfigured. The level of sensitivity for detecting warning sounds may also be adjusted (504) by the user. In the event that a warning sound is detected (506) by the processor 12, the warning handler 48 is triggered.

In yet another embodiment, the warning sounds may be recorded by the HPD 10, and the recorded data can be streamed to a mobile or a central processing unit and from there they can be transmitted through the Internet and/or Internet-of-Things to a cloud and/or a central server for further development and training of the signal detection. Part of or the entire of the signal processing algorithm may be upgraded based on recorded feedback data, and the new codes can be rewritten automatically as an update to any of the HPD 10 that are connected to a mobile companion application and from there connected to the Internet.

In one embodiment, the HPD 10 can detect a pattern of sounds and perform an action related to a warning related to the sounds. These patterns of sounds can be three whistles or a particular phrase like “Man Down” and the action can be to call the pre-set emergency contact or 911 or calling or paging the lead of operation. It can also be to turn on the lights associated with the HPD 10 (e.g. the notification area 22) and make them flash with different colors to be able to easily spot the victim or user in need. This can be in particular useful for incapacitated user in need.

In one embodiment, the warning handler 48 may transmit (508) a message to the external processor 16, with the message containing information regarding the detected warning sounds. The external processor 16 may be located on another device external to the HPD 10, such as the mobile device 60. The external processor 16 may carry on additional processing on the detected warning sounds. The external processor 16 may have access to information and data not available to the processor 16 (such as location information, etc.). This information or data may affect whether the presence of the warning sound should be alerted to the user. For example, if the external processor 16 has access to location information about the user and the external processor 16 determines that the user is in an area that does not need to be subject to warning sound detection, the external processor 16 may override detection of the warning sound and not alert the user

Based on the external processor 16 processing of the warning sound, the external processor 16 may (or may not) cause (510) the notification module 76 to be triggered.

Alternatively, in the event that the external processor 16 is not involved, the warning handler 48 may directly trigger (512) the notification module 76. The notification module 76 may then cause (514) the notification to be provided to the user (e.g. through auditory signal, vibratory feedback, etc.).

The external processor 16 (or the application 106) may be used to perform real-time or ahead of time noise cancellation processes using variation of noise cancellation and speech enhancement algorithm if the processor 12 of the HPD 10 is not sufficient or is in power saving mode. The external processor 16 may also be used to process coding or decoding the data if the processor 12 of the HPD 10 is not sufficient or is in power saving mode.

Referring to FIG. 7, which shows a block diagram of some of the components of the HPD 10, the processor 12 receives input from the microphones 14 in the form of electrical signals (converted from audio signals). The electrical signals are processed by the processor 12. In the event that a warning sound is detected, the processor 12 may send a notification to the warning handler 48, which in turn may (as discussed above) send a notification to the notification module 76 for communication to the user (e.g. by auditory signals, vibratory feedback, etc.).

In the event of detecting a potential alert, the processor 12 might not send any signal to the warning handler 48, if the detected warning sound is too far, or in the line of sight of the user (can realize through directions using proximity sensors 58 (of FIG. 5) or array of the microphones 14) or if the user already moved away or if the user disabled the system 5 from notifying him/her from such type of the warnings.

In addition, the processor 12 may also communicate with an audio controller 78 to adjust and/or control the volume and/or content of the auditory output transmitted to the earpad 18 or the earbud 34.

The battery 42 may provide power to the HPD 10. The battery 42 may be rechargeable. The HPD 10 may also comprise a power management module 80, including software, hardware, or combination thereof, for controlling power provided to the microphones 14, the processor 12, and/or the audio controller 78.

In another embodiment, voice may be captured by the HPD 10, and noise cancellation and coding can be applied to it by the processor 12 or the mobile device 60. The resultant data may be sent to the central gateway 54 that other users are also connected to. If the users are set to ‘listen” to a particular channel or user, the users would be able to hear the sound from anybody that is registered to those channels or the particular user. This may be referred to as voice communication as URPTT (“Unlimited Range Push To Talk”). URPTT can be a two-way or one-way channel. URPTT may use Internet, Wi-Fi, Internet of Things, or Bluetooth to connect to all users.

In yet another implementation, the voice communication can be transferred between two or more of the HPD 10 without the use of the mobile device 60. This may be referred to as SRPTT (for “Short Range Push To Talk”). Voice will be recorded by each of the HPD 10, after potential noise cancellation and coding algorithms are applied. The resultant data will be sent to one or more recipients that are set by the mobile or through pairing process, through BLE, WIFI, or Bluetooth. Recipients can engage in a conversation by pushing a Push-To-Talk (“PTT”) button 81 provided on the HPD 10.

In embodiments, to start either the URPTT or SRPTT process, the PTT button 81 may be pressed and held by the user. While the user is holding the PTT button 81 (pressed), the user can talk and his or her conversation will be recorded and transmitted to the other users (through their HPDs 10). In yet another implementation, the user may push the PTT button 81 twice (or thrice) in rapid succession and start the conversation. In some embodiments, the user does not need to hold down the PTT button 81 to start the conversation. This method can be referred as hands-free PTT. In some embodiments, to stop the conversation, the user may press the PTT button 81 one time. Both SRPTT and URPTT may enable communication between HPDs 10 that are “registered” with the same organization. That means that if a person found one of the HPD 10 and is part of the same organization, that person cannot tap over the conversation.

In various embodiments, to make the SRPTT and URPTT processes secure, each of the HPD 10 has to be first registered. The registration process can be conducted by connecting to another one of the HPD 10 (e.g. using BLE connectivity). In embodiments, the user should create a password for each of the HPD 10 or security can be enabled by face ID, biometric, or other identifying technology. The user preferably needs to enter the password to connect the HPD 10 to the application 106 to set up and initialize the URPTT and SRPTT communication groups. During registration, the processor 12 may be configured to send some unique characteristics of the HPD 10 to the registering device (which may be the mobile device 60) and it can be later sent to a central server 7 to record and use as identification criteria for the HPD 10. These unique criteria may include one or more of the device IMEL, serial number, firmware revision, code name, and any secret code that is registered in the HPD 10 or any combination of those. In embodiments, when the user connects to the HPD 10 using the password, the end user app would compare that with what is recorded on the server 7 and if correct, it would allow the user to configure and listen to URPTT and SRPTT groups.

In embodiments, the SRPTT process will always connect to any nearby users wearing other ones of the HPD 10. In addition, the HPD 10 may continue to refresh to search for the new ones of the HPD 10 in range. It is possible to customize (e.g. using the application 106) the range in which the SRPTT process is used to connect nearby ones of the HPD.

The SRPTT process can use different mechanisms to estimate the proximity of other users. Such method can be through using Bluetooth, Wi-Fi, or other signal strength power, where lower signal strength can represent users in farther distances. For example, the processor 12 may be provided information regarding signal strength from external sources (e.g., a signal strength meter IC) or using a Received Signal Strength Indicator (RSSI) index within the HPD 10. The SRPTT process can also use other mechanisms to estimate the distance between users, such as the proximity sensors 58 located on the HPD 10.

In one embodiment, one of the HPDs 10 (e.g. HPD 10a) may connect to another one of the HPDs 10 (e.g. HPD 10b) using some electromagnetic-based communications standard (e.g. Bluetooth, Bluetooth Low Energy, Zigbee, or the like). The HPD 10a may also be configured to send a pattern of sound in the form of a short pulse of sounds containing one or more frequency components to the HPD 10b. A distance between the HPD 10a and the HPD 10b may be estimated as follows. At or around the time the pulse of sound is transmitted by the HPD 10a, the HPD 10a is also configured to transmit (using the electromagnetic-based communications standard) a time stamp comprising the time the sound pulse is transmitted. It is also possible to calculate and take into account the delay that it takes to generate the sound pulse into the time stamp. When the HPD 10b receives the sound pulse, the HPD 10b is able to determine the time at which the pulse is received. By comparing the time at which the pulse is received with the time in the time stamp (indicating when the pulse was transmitted), and based on the speed of sound in air, the HPD 10b is able to estimate the distance between the HPD 10a and the HPD

In another embodiment, the HPD 10a is configured to record the time that the sound pulse is transmitted. When the HPD 10b receives the sound pulse, the HPD 10b is configured to transmit an acknowledgment to the HPD 10a (using the electromagnetic-based communications standard). When the HPD 10a receives the acknowledgment, the HPD 10a is configured to compare the time at which the acknowledgement is received with the time at which the sound pulse is transmitted. The difference between the two times is the time of flight (of the sound pulse). Using this time of flight, it is possible for the HPD 10a to estimate the distance between the HPD 10a and the HPD 10b. This approach does not require that the HPD 10a and HPD 10b run on synchronous clocks.

It is also possible to implement the above distance estimation methods using other devices other than the HPDs 10. For example, it is possible to use a first handheld mobile device that is configured to generate sound and that is configured to communicate using an electromagnetic-based communications standard (e.g. Bluetooth, Bluetooth Low Energy, Zigbee, or the like). By also using one or more second handheld mobile devices that are configured to receive sound (e.g. through a microphone) and that are configured to communicate using the electromagnetic-based communications standard, the first handheld mobile device is able to estimate a distance between it and one or more of the second handheld mobile devices using the methods discussed above.

In some embodiments, the SRPTT process may only connect to a limited number of users. The SRPTT process may use a group identification tag and/or estimated users' distances as two factors to choose which limited users would be selected to be connected. For example, the SRPTT process may connect the HPD 10 to the nearest five users that share the same particular group identification tag. The group identification tag may be set during registration.

To ensure that the SRPTT and URPTT processes and streaming have substantially maximum quality of sound, the processor 12 can execute a programmable coder and decoder algorithm that adjusts the data compression of the raw signals. When the number of connected users increases or the users are located in farther distances (i.e. more lossy channel), the processor 12 may be configured to increase the compression ratio, and when number of users decreases or the distances are shortened, the processor 12 may be configured to use lower compression ratio and higher sound quality.

The mobile device 60 may remember the password, and if the HPD 10 corresponds to the mobile device 60 (using the same unique criteria of each HPD 10 hardware), the mobile device 60 will not ask for the password, at least for a particular period of time.

In various embodiments, during registration of each of the HPD 10, the application 106 may automatically generate a software tag to devices in the same group of employees or affiliated users to identify them from each other. This tag maybe advertised by the HPD 10 after SRPTT and URPTT activation (by the user after entering the password) and the user can listen and connect by SRPTT to other users that carrying the similar tag or tags. This is useful to ensure that the SRPTT process is only working for same organization's employees or for particular groups inside a company (e.g. fueling crew).

The application 106 may allow the user to select from a number of voice channels that the user can choose to listen to. When the user selects one or more channels, the user would be notified about an incoming conversation if the user is not wearing the HPD 10 or if the HPD 10 is disconnected from mobile device 60. This notification can be in the form of an alarm and/or a written notification message to the mobile device 60.

In some embodiments, if the user is wearing the HPD 10 and chooses to listen to one or multiple channels/groups, when an incoming conversation arrives in any of these channels, the user can hear them. If the user choses to reply, the user can press the PTT button 81, and the application 106 will automatically send the user's response (vocal) to the last channel from which the user received conversation. Alternatively, the HPD 10 may be configured such that if the user presses the PTT button 81 within a certain amount of time, the application 106 will send the user's response to the last channel from which the user received conversation. If the user presses the PTT button 81 after a certain amount of time, the application 106 will send the user's response to a preprogrammed channel.

In another embodiment, the HPD 10 may be configured such that the PTT button 81 does not need to be pressed to initiate conversations. In this embodiment, the HPD 10 may be configured to detect voice (i.e. from a person talking). If the HPD 10 detects one or more voices, the HPD 10 may be configured to automatically commence a conversation.

The application 106 can transfer the geographical information of the user to the server 7 using an onboard GPS (global positioning Systems) on the HPD 10 or by GPS or A-GPS on the mobile device 60. This data can be used to determine, for example, if some users are still on site when the site is to be evacuated. The data may also be used for localizing uses, annotating users that are not moving and may be injured, or identifying the location of users with specific conditions (e.g. coronavirus, etc.).

In one embodiment, the HPD 10 may be configured to detect when other users with other ones of the HPD 10 are located within a certain distance. That can be done either using GPS system above or RSSI RF power levels that can be measured by the HPD 10. If the measured RSSI values are higher than some threshold that indicates that the other ones of the HPD 10 are closer than certain threshold. This may be useful for social distancing purposes. For example, the HPD 10 may be configured to provide an alert to the user if the HPD 10 detects that another one of the HPD 10 is within two meters.

In another embodiment, a number of the HPDs 10 may be used to enhance pedestrian safety. It is important for a driver of large vehicles (e.g. industrial vehicles, forklifts, etc.) to be aware of their surroundings. However, due to the large size of the vehicles, impaired visibility, and/or loud environments, it is not always possible for the driver to be fully aware of their surroundings. In this embodiment, the driver, using one of the HPDs 10, is able to set up a virtual “fence” around the HPD 10 of the driver. Once the “fence” has been set up, the HPD 10 of the driver is configured to generate an alert when it detects another of the HPDs 10 enters the “fence”. The “fence” may have a range that is configurable. It may be a relative range (e.g. short, medium, or long range) or it may be a set value (e.g. 10 meters, 20 meters, etc.).

For example, if the range of the “fence” is configured to be “short”, the HPD 10 of the driver may be configured to send a signal to other nearby ones of the HPDs 10 that indicates the presence of the “fence”. The signal may include information indicating the range of the “fence”. The other ones of the HPD 1s are able to estimate a distance between them and the HPD 10 of the driver, using RSSI or other methods discussed above. If one of the HPD 10s determines that it is within the range of the “fence”, that HPD 10 may generate an alert to the user wearing the HPD 10. In addition, that HPD 10 may also transmit an alert to the HPD 10 of the driver, which may in turn be configured to generate an alert to the driver.

Furthermore, the HPD 10 of the driver may also be configured to receive signals from other ones of the HPDs 10. The HPD 10 of the driver may be configured to estimate a distance between it and the other ones of the HPDs 10, using RSSI or other methods discussed above. If the HPD 10 of the driver determines that one of the other ones of the HPDs 10 is within the range of the “fence”, the HPD 10 of the driver may generate an alert to the driver. In addition, the HPD 10 of the driver may also transmit an alert to the HPD 10 of the other ones of the HPDs that is within the range of the “fence”, which may in turn be configured to generate an alert to the user wearing that HPD 10.

Alternatively, if the range of the “fence” is configured to be “medium”, the HPDs 10 may use one or both of RSSI or a global navigation satellite system (e.g. GPS, Galileo, or the like) to estimate a distance between the HPD 10 of the driver and other ones of the HPDs 10. Each of the HPDs 10 may be configured to retrieve a position of the HPD 10 (e.g. using GPS). The HPD 10 of the driver is configured to periodically transmit a signal comprising a current position of the HPD 10 of the driver. The signal may also comprise a range of the “fence”. The other ones of the HPDs 10, upon receiving the signal, are able to estimate a distance to the HPD 10 of the driver, and determine whether it is within the range of the “fence” set up by the HPD 10 of the driver. If it is determined that one of the HPDs 10 is within the range of the “fence”, that HPD 10 may be configured to generate an alert for the user of that HPD 10. Similarly, the HPD 10 of the driver may also estimate distances to other ones of the HPDs 10, using for example RSSI or other methods discussed above. This provides a level of redundancy. The use of RSSI may be used in combination with a global navigation satellite system. The estimation of distances using RSSI may also override the estimation of distances using a global navigation satellite system.

If the range of the “fence” is configured to be “long”, the HPDs 10 may be configured to use the global navigation satellite system to estimate distances between the HPD 10 of the driver and other ones of the HPDs 10. However, even when the “fence” is configured to be “long” the techniques used for when the “fence” is configured to be “medium” or “short” may still be enabled in order to provide a level of redundancy. For example, RSSI may still be active, even when the range of the “fence” is configured to be “long”.

The HPD 10 may also be configured to use sound detection algorithms to detect the backup alerts. Based on the sound level, it is also possible to calibrate the HPD 10 to derive some distance estimations. By using a combination of methods, a more reliable and faster process for monitoring distances can be carried out.

On top of the SRPTT and URPTT processes, the ports 38 may include a sound port (e.g. 32 mm jack or other audio input/output ports) that connects to the conventional one-way or two-way radios. The user may change the setting to send the voice by pressing the PTT button 81 from the HPD 10, and the processor 12 may apply denoising algorithm before transferring sounds to the radio systems.

In case of receiving sound from conventional radio systems, the processor 12 may apply one or more denoising algorithms to receiving sounds before playing it on earmuff or earplug speakers. In addition, the processor 12 may automatically detect the incoming radio voice from external radio devices and subsequently automatically play that to the users' ears.

For example, the processor 12 may be configured to apply one or more denoising

algorithms when it detects that the background noise in the sound received is greater than a set amount. The denoising algorithms allow the sound received be more natural-sounding. In order to determine when to apply the denoising algorithms, the processor 12 can apply one of several methods, including, for example, using the root mean square of a certain frame of sound and determining whether it is above set threshold.

Furthermore, the processor 12 may also be configured to apply dynamic gain control on the sound received. For example, the processor 12 may be configured to continually calculate the power of the incoming sound. If it is less than a set threshold, the processor 12 may be configured to apply gain (i.e. amplify it), and if it is greater than a set threshold, the processor 12 may be configured to attenuate it.

The processor 12 in the HPD 10 or the external processor 16 can detect other users' sneezing and coughing sounds and in combination with data coming from temperature sensors 83 embedded on the earpads 18 or the earbuds 34 or other parts of the user, detect whether the user might be infected with influenza or any other virus. This data may be transferred to the central gateway 54 or the server 7 for further processing and for monitoring the spread of the virus.

The user may reach customer support through the application 106. By adding the information about the nature of support required, the application 106 is able to transmit the user's request to a helpdesk, and a member of support team can assist the user.

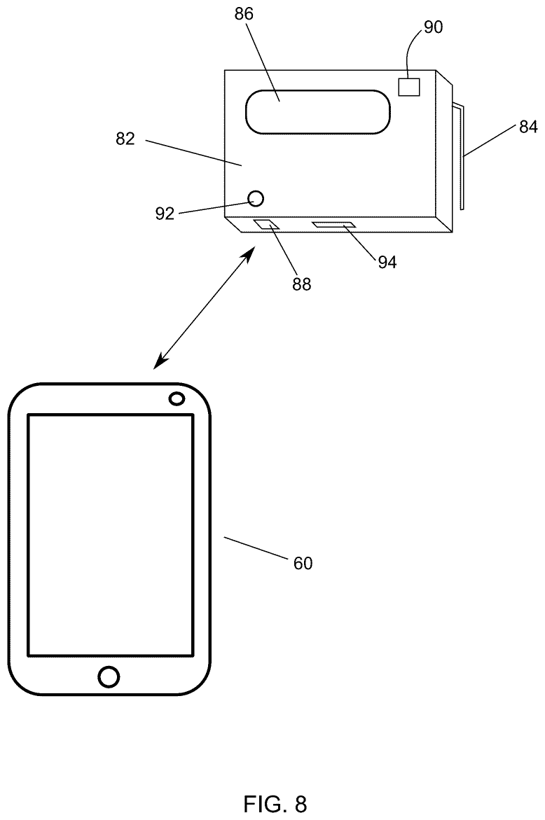

In another embodiment, referring to FIG. 8, a tag 82 may be provided. The tag 82 preferably comprises a clip 84 that is adapted to removably attach the tab 82 to the user's clothing. For example, the clip 84 may comprise adhesive material for attaching the tag 82 to the clothing, or the clip 84 may have some mechanical mechanisms for attachment to the clothing. The tag 82 comprises a tag display 86 for displaying information to the user and a tag transceiver 88 for wireless communications. The tag transceiver 88 may communicate with mobile device 60 carried by the user. In such an embodiment, the mobile device 60 may have installed upon it a mobile application to coordinate communications between the mobile device 60 and the tag 82. The wireless communications may be done through Bluetooth, Wi-Fi, or the like.

The tag 82 comprises a tag processor 90 and one or more tag microphones 92. The tag microphones 92 are configured to detect and capture sound external to the tag 82 and to convert them into electrical representations for processing by the tag processor 90. Using techniques described earlier, the tag processor 90 is configured to detect if the electrical representations captured by the tag microphones 92 correspond to specific predetermined sounds. For example, if the electrical representations correspond to a human voice saying “behind”, the tag processor 90 may be configured to cause an alert to be raised. This could indicate a possible cyclist or person behind and approaching the user. The specific predetermined sounds may include other words (e.g. “stop”, “watch out”, etc.) or other non-verbal sounds (e.g. a bicycle bell, a siren, etc.).

The alert may cause tag vibrators 94 on the tag 82 to activate, causing vibrations that may be felt by the user. Alternatively, the alert may be transmitted by the tag transceiver 88 to the mobile device 60. This may cause the mobile device 60 to, for example, pause the playback of music or other audio so that the user can now hear external sounds more clearly.

The tag display 86 may display information to the user regarding the alert. In addition, the tag display 86 may be configured to display other information when the alert is not in effect. For example, the tag display 86 may be configured to display the title or artist for music currently playing on the mobile device 60.

In one embodiment, one or more of the HPDs 10 may be connected through the Internet to the server 7. The HPDs 10 may be configured to transmit their location to the server 7. The server 7 may be configured to provide an interface for allowing an administrator to view the location of the HPDs 10 and any virtual “fences” that are set up by any of the HPDs 10. The interface may be accessed using a web application or the like. The server 7 may also be configured to display on the interface any alerts generated by the HPDs 10. The server 7 may also be configured to generate alerts if it detects that other ones of the HPDs 10 are within the “fence” set up by one of the HPDs 10.

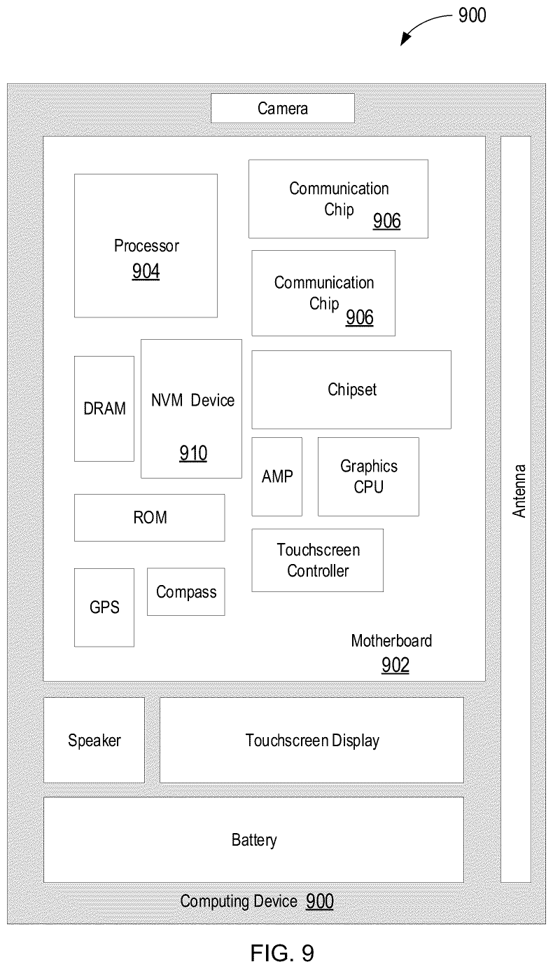

FIG. 9 illustrates a computing system including a computing device 900 that may be used to implement various aspects of the embodiments of the present disclosure. In some embodiments, computing device 900 or components of computing device 900 includes, e.g., mobile phone 60 of FIGS. 1, 2, 4, and 8 or remote computer or a server (e.g., that includes server 7 of FIG. 1) communicatively couples to mobile phone 60 and/or HPD 10.

In embodiments, computing device 900 houses a board 902, such as, for example, a motherboard. The board 902 may include a number of components, including but not limited to a processor 904 and at least one communication chip 906. The processor 904 is physically and electrically coupled to the board 902. In some implementations, the at least one communication chip 906 is also physically and electrically coupled to the board 902. In further implementations, the communication chip 906 is part of the processor 904.

Depending on its applications, computing device 900 may include other components that may or may not be physically and electrically coupled to the board 902. These other components include, but are not limited to, volatile memory (e.g., DRAM), non-volatile memory (e.g., ROM), flash memory, a graphics processor, a digital signal processor, a crypto processor, a chipset, an antenna, a display, a touchscreen display, a touchscreen controller, a battery, an audio codec, a video codec, a power amplifier, a global positioning system (GPS) device, a compass, an accelerometer, a gyroscope, a speaker, a camera, and a mass storage device (such as hard disk drive, compact disk (CD), digital versatile disk (DVD), and so forth).

The communication chip 906 may enable wireless communications for the transfer of data to and from the computing device 900, including, e.g., between computing device 900 and HPD 10 of FIGS. 1-4. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. The communication chip 906 may implement any of a number of wireless standards or protocols, including but not limited to Wi-Fi (IEEE 802.11 family), WiMAX (IEEE 802.16 family), IEEE 802.20, long term evolution (LTE), Ev-DO, HSPA+, HSDPA+, HSUPA+, EDGE, GSM, GPRS, CDMA, TDMA, DECT, Bluetooth, derivatives thereof, as well as any other wireless protocols that are designated as 3G, 4G, 5G, and beyond. The computing device 900 may include a plurality of communication chips 906. For instance, a first communication chip 906 may be dedicated to shorter range wireless communications such as Wi-Fi and Bluetooth and a second communication chip 906 may be dedicated to longer range wireless communications such as GPS, EDGE, GPRS, CDMA, WiMAX, LTE, Ev-DO, and others.

The processor 904 of the computing device 900 includes an integrated circuit die packaged within the processor 904. The term “processor” may refer to any device or portion of a device that processes electronic data from registers and/or memory to transform that electronic data into other electronic data that may be stored in registers and/or memory.

In various implementations, the computing device 900 may be a laptop, a netbook, a notebook, an ultrabook, a smartphone, a tablet, a personal digital assistant (PDA), an ultra mobile PC, a mobile phone (e.g., mobile phone 60 of FIGS. 1, 2, 4, and 8)), a desktop computer, a server such as the server 7 in FIG. 1 (e.g., a data center server or high-performance server). In further implementations, the computing device 900 may be any other electronic device that processes data.

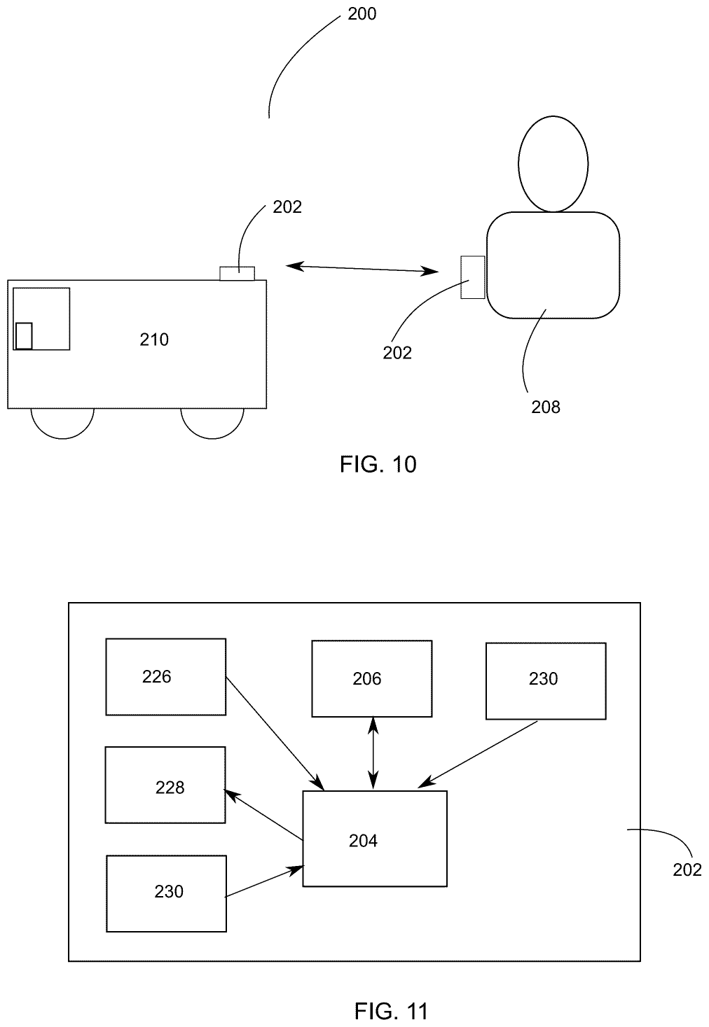

Referring to FIGS. 10 and 11, in some embodiments, a system 200 for improving pedestrian safety comprises a plurality of communications devices 202 that are configured to communicate wirelessly. Each of the communications devices 202 comprises a communications device processor 204 and a communications device transceiver 206 configured for wireless communications with other ones of the communications devices 202. The communications devices 202 may be carried by users 208 (such as pedestrians) and located on vehicles 210 (such as trucks, industrial vehicles, forklifts, etc.). In some embodiments, the communications devices 202 may comprise some of the components of the HPDs 10.

It is important for drivers of the vehicles 210 to be aware of their surroundings. The drivers of the vehicles 210, using one of the communications devices 202 is able to set up a virtual “fence” around the communications device 202 of the vehicle 210. Once the “fence” has been set up, the communications device 202 of the vehicle 210 is configured to generate an alert when it detects another of the communications device 202 entering the “fence”. The “fence” may have a range that is configurable. It may be a relative range (e.g. short, medium, or long range) or it may be a set value (e.g. 10 meters, 20 meters, etc.).

For example, if the range of the “fence” is configured to be “short”, the communications device 202 of the vehicle 210 may be configured to send a signal to other nearby ones of the communications device 202 that indicates the presence of the “fence”. For example, a “short” range for the “fence” may be considered to be less than 10 meters. The signal may include information indicating the range of the “fence”. The other ones of the communications devices 202 are configured to estimate a distance between them and the communications device 202 of the vehicle 210, using RSSI or other methods discussed above. If one of the communications device 202 determines that it is within the range of the “fence”, that one of the communications device 202 may generate an alert to the user 208 carrying that one of the communications device 202. In addition, that one of the communications device 202 may also be configured to transmit an alert to the communications device 202 of the vehicle 210, which may in turn be configured to generate an alert to the driver of the vehicle 210.

Furthermore, the communications device 202 of the vehicle 210 may also be configured to receive signals from other ones of the communications device 202 (e.g. the communications device 202 carried by the users 208). The communications device 202 of the vehicle 210 may be configured to estimate a distance between it and the other ones of the communications device 202, using RSSI or other methods discussed above. If the communications device 202 of the vehicle 210 determines that one of the other ones of the communications device 202 is within the range of the “fence”, the communications device 202 of the vehicle 210 may be configured to generate an alert to the driver of the vehicle 210. In addition, the communications device 202 of the vehicle 210 may also be configured to transmit an alert to the other ones of the communications devices 202 that is within the range of the “fence”, which may in turn be configured to generate an alert to the user(s) 208 carrying such communications device(s) 202.

Alternatively, if the range of the “fence” is configured to be “medium”, the communications device 202 may use one or both of RSSI or a global navigation satellite system (e.g. GPS, Galileo, or the like) to estimate a distance between the communications devices 202 of the vehicle 210 and other ones of the communications devices 202. For example, a “medium” range for the “fence” may be considered between 10 and 20 meters. Each of the communications devices 202 may comprise a communications device GPS 212 and may be configured to retrieve a position of the communications devices 202 (e.g. using the communications device GPS 212). The communications devices 202 of the vehicle 210 is configured to periodically transmit a signal comprising a current position of the communications device 202 of the vehicle 210. The signal may also comprise a range of the “fence”. The other ones of the communications devices 202, upon receiving the signal, are able to estimate a distance to the communications device 202 of the vehicle 210, and determine whether it is within the range of the “fence” set up by the communications device 202 of the vehicle 210. If it is determined that one of the communications device 202 is within the range of the “fence”, that communications device 202 may be configured to generate an alert for the user 208 of that communications device 202. Similarly, the communications device 202 of the vehicle 210 may also estimate distances to other ones of the communications device 202, using, for example, RSSI or other methods discussed above. This provides a level of redundancy. The use of RSSI may be used in combination with a global navigation satellite system. The estimation of distances using RSSI may also override the estimation of distances using a global navigation satellite system.

If the range of the “fence” is configured to be “long”, the communications device 202 may be configured to use the global navigation satellite system to estimate distances between the communications device 202 of the vehicle 210 and other ones of the communications device 202. For example, a “long” range for the “fence” may be considered greater than 20 meters. However, even when the “fence” is configured to be “long”, the techniques used for when the “fence” is configured to be “medium” or “short” may still be enabled in order to provide a level of redundancy. For example, RSSI may still be active, even when the range of the “fence” is configured to be “long”.

The communications device 202 may also be configured to use sound detection algorithms to detect the backup alerts. Based on the sound level, it is also possible to calibrate the communications device 202 to derive some form of distance estimations. By using a combination of methods, a more reliable and faster process for monitoring distances can be carried out.

In other embodiments, machine learning techniques may also be used with RSSI to identify a pattern between different channels' strengths at different distances and angles relative to the communications device 202 of the vehicle 210. One way of doing so is to create a labelled dataset, with the labelled dataset comprising data that includes, but is not limited to: (1) the channel (e.g. for BLE) the RSSI is measured from; (2) the RSSI value of the communications devices 202; (3) time series data (e.g. past RSSI values, channels, and timestamps for data, etc.); (4) BLE events (e.g. connection events, advertisement packets received, etc.); (5) GPS data; and (6) aggregates of the above data (e.g. average or variance of raw values, counts of BLE events, etc.).

With the labelled dataset created, it is possible to create a trained model using supervised machine learning techniques. The model may be for classification (e.g. determining if a distance between the communications device 202 of the vehicle 210 and other ones of the communications devices 202 is between, for example, 3 and 5 meters or 5 and 10 meters), or if possible, for regression (e.g. predicting an actual distance, such as 3.4512 meters).

Depending on the model (and possibly after pruning if applied to a decision tree model or neural network), it is possible that not all of the data in the dataset will be used. The unused data may not need to be collected during runtime, saving computational resources.

In some embodiments, the model may be executed on the communications devices 202 (e.g. by the communications device processors 204), which may limit the number of parameters in the model. Alternatively, the model may be executed on a connected device, such as the mobile device 60. The connected device may allow for larger ones of the models to be executed and may also take up some of the BLE bandwidth.

Using connections and advertisements for transferring and measuring RSSI data allows for faster decision-making when identifying if an object is getting too close (e.g. such as one of the vehicles 210 to one of the users 208). Using RSSI data collection on connection events may provide one data point per connection interval. For example, in the case of BLE connections, 7.5 ms may be the lowest connection interval, but this may range from 7.5 ms to 4 s, depending on the circumstances.

By calibrating the gain from each of the channels (instead of averaging all of the RSSI values), it is possible to calibrate the channels and always receive useful RSSI data from all of the channels. Each of the channels may have different gains (compared to each other) as a result of different impedance matching, housing, and other environmental factors.

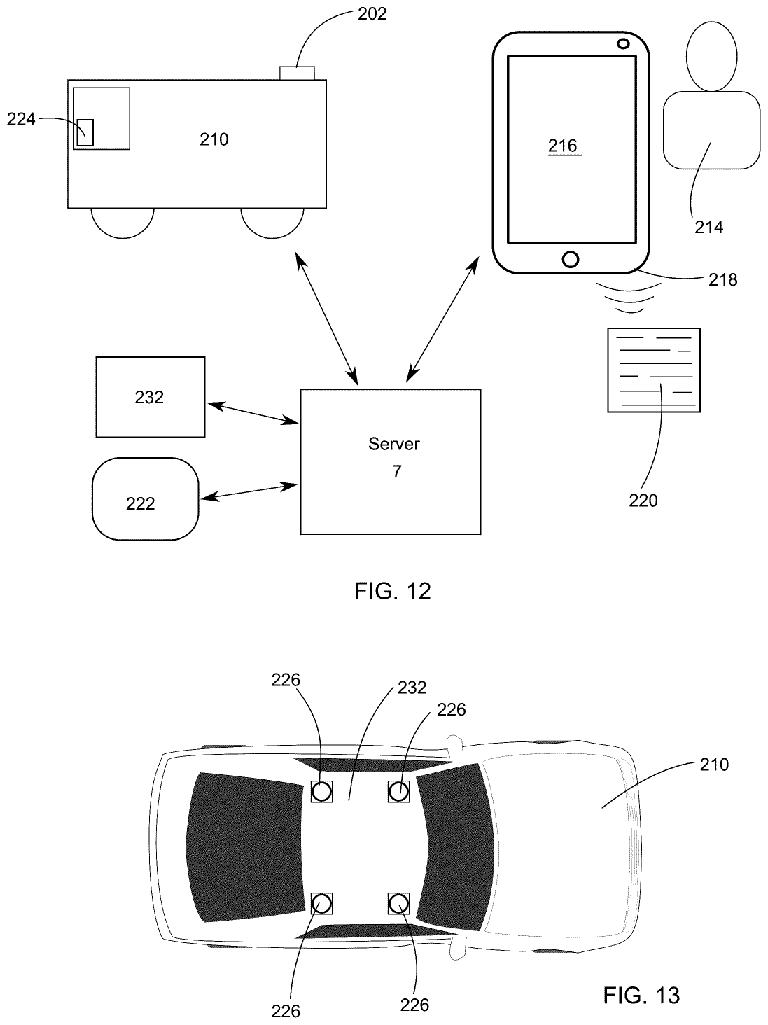

Referring to FIG. 12, in some embodiments, a visitor 214 may have a visitor device 216, which may be a smartphone, a mobile device, or the like. The visitor device 216 comprises a scanning module 218. The scanning module 218 may include a camera or the like. The visitor 214 may be provided with a code 220 that is capable of being scanned with the scanning module 218. The code 220 may include a quick-response (QR) code. When the code 220 is scanned by the scanning module 218, the visitor device 216 is configured to download and execute an application on the visitor device 216. The server 7 is configured to communicate with the visitor device 216 and the communications devices 202 and is further configured to determine a geographical location of the visitor device 216 and the communications devices 202, using, for example, GPS data received from the visitor device 216 and the communications devices 202.

The server 7 is configured to monitor the locations of the visitor device 216 and/or the communications device 202 on the vehicles 210 and to determine a proximity of the visitor device 216 to the communications devices 202 on the vehicles 210. The locations of the visitor device 216 and/or the communications devices 202 on the vehicles 210 may be displayed for viewing through an interface 222. The interface 222 may be a web interface or the like.

In other embodiments, the visitor devices 216 and the communications devices 202 are configured to determine a distance between itself and other ones of the visitor devices 216 and/or the communications devices 202 (rather than having the determination conducted by the server 7). The visitor devices 216 and the communications devices 202 are configured to transmit their respective location information to the server 7, which in turn relays the location information to other ones of the visitor devices 216 and/or the communications devices 202. The location information is processed by the respective visitor devices 216 and/or the communications devices 202 to determine a distance between the respective visitor device 216 or the communications devices 202 and other ones of the visitor devices 216 and/or the communications devices 202. In other words, the distance or proximity processing is performed by the visitor devices 216 and the communications devices 202 themselves (rather than by the server 7).

The server 7 may be configured to generate notifications to the visitor device 216 and/or the communications devices 202 on the vehicles 210 if they are too close to each other (e.g. less than a certain threshold of distance). The notifications may be an auditory message (e.g. configured to be played by the visitor device 216) or a visual message (e.g. configured to be displayed by the visitor device 216). The notification may also include information regarding the relative locations of the visitor device 216 and/or the communications devices 202 of the vehicles 210.

In some embodiments, the interfaces 222 may comprise software clients 232. The server 7 may be configured to also allow for access by one or more software clients 232. Upon connection by the software clients, the server 7 may be configured to display on the software clients 232 a map display showing the location of the visitor devices 216 and the communications devices 202 (i.e. on the vehicles 210) registered for a particular location. The software clients 232 may also receive notifications when the visitor devices 216 and/or the communications devices 202 on the vehicles 210 are too close to each other (i.e. less than a certain threshold of distance). A historical log of the notifications may be maintained by the server 7 to allow for subsequent investigations or audits into potential safety incidents.

In some embodiments, one or more of the vehicles 210 may be equipped with a vehicle head unit 224 that is in data communications with the communications device 202 on the vehicle 210. The vehicle head unit 224 may be configured to display the relative locations of the visitor device(s) 216 (e.g. relative to the vehicle 210). The vehicle head units 224 may also be configured to play an auditory message or display a visual message if the server 7 determines that the visitor device 216 is within the threshold distance of the communications device 202 of the vehicle 210.

In some embodiments, if the visitor device 216 and/or the communications devices 202 on the vehicles 210 comprise magnetometers, more relatable feedback may be provided to the visitor 214 and/or the drivers of the vehicles 210. For example, the vehicle head unit 224 may be configured to state, “Pedestrian on the back left”, or the visitor device 216 may be configured to state, “Driver in front right”. Furthermore, the vehicle head unit 224 for a particular one of the communications devices 202 may be configured to display a map in which other ones of the visitor devices 216 and/or the communications devices 202 on other ones of the vehicles 210 are oriented and positioned relative to the particular one of the communications devices 202. In other words, if the particular one of the communications devices 202 turns in one direction, the vehicle head unit 224 is configured to re-orient the display of the positions of the other ones of the visitor devices 216 and/or the communications devices 202 on the other ones of the vehicles 210 accordingly.

In some embodiments, the communications devices 202 may be used to detect particular sounds of interest (e.g. sirens). The communications devices 202 may comprise a plurality of communications device microphones 226. The communications devices 202 may be configured to determine a directionality and movement of a source of the particular sounds of interest. For example, the communications device 202 may comprise a communications device speaker 228 that may be configured to play an audible message (such as “Ambulance approaching!”) that advises whether the source of the particular sound of interest is approaching or moving further away. The communications device 202 may also be configured to only generate the audible message when the source of the particular sound of interest is approaching (and not, for example, when the source is moving further away).

In some embodiments, the communications devices 202 may be integrated with the vehicles 210. In such embodiments, the communications device microphones 226 may be placed at different locations on the vehicle 210. For example, FIG. 13 depicts one possible arrangement, where the communications device microphones 226 are located proximate to one or more corners of a roof 232 of the vehicle 210. It is understood that other arrangements of the communications device microphones 226 are also possible. By locating the communications device microphones 226 at different places on the vehicle 210, a directionality of the source of the particular sounds of interest may also be determined. The communications device processor 204 may be configured to detect particular sounds of interest (e.g. sirens) using the communications devices microphones 226 and may be configured to turn off any music playing within the vehicle 210 (e.g. from the communications device speakers 228 or from other speakers within the vehicle 210) if particular sounds of interest are detected. The communications device processor 204 may further be configured to play the particular sounds of interest (e.g. using the communications device speakers 228 or other speakers within the vehicle 210) in order to further alert the driver of the vehicle 210. Alternatively, or additionally, the communications device processor 204 may be configured to play an audible message (e.g. using the communications device speakers 228 or other speakers within the vehicle 210) notifying the driver of the vehicle 210. The audible message may be, for example, an indication of the direction of the source of the sound of interest and whether it is approaching or moving further away. Such audible messages may be in the form of “Approaching ambulance in the rear” or “Firetruck approaching from the front” or the like.

In some embodiments, the communications device processor 204 may be configured to cause only certain ones of the speakers in the vehicle 210 to play the particular sounds of interest, depending on the directionality of the particular sounds of interest. For example, if the particular sound of interest was determined by the communications device processor 204 to be originating from the front, driver-side of the vehicle 210, the communications device processor 204 may be configured to only play the particular sound of interest using the front, driver-side speaker in the vehicle 210, thus providing a directional cue to the driver of the vehicle 210. The communications device processor 204 may be configured to directly map sounds from particular ones of the communications device microphones 226 to particular ones of the speakers in the vehicle 210. For example, if the particular sound of interest was determined by the communications device processor 204 to be mainly detected by the front, driver-side one of the communications device microphones 226, the communications device processor 204 may be configured to map the particular sound of interest to be played by the front, driver-side one of the speakers in the vehicle 210.

Alternatively, or additionally, the communications device processor 204 may be configured to cause the vehicle head unit 224 to display an approximate and/or relative position of the source of the sound of interest.

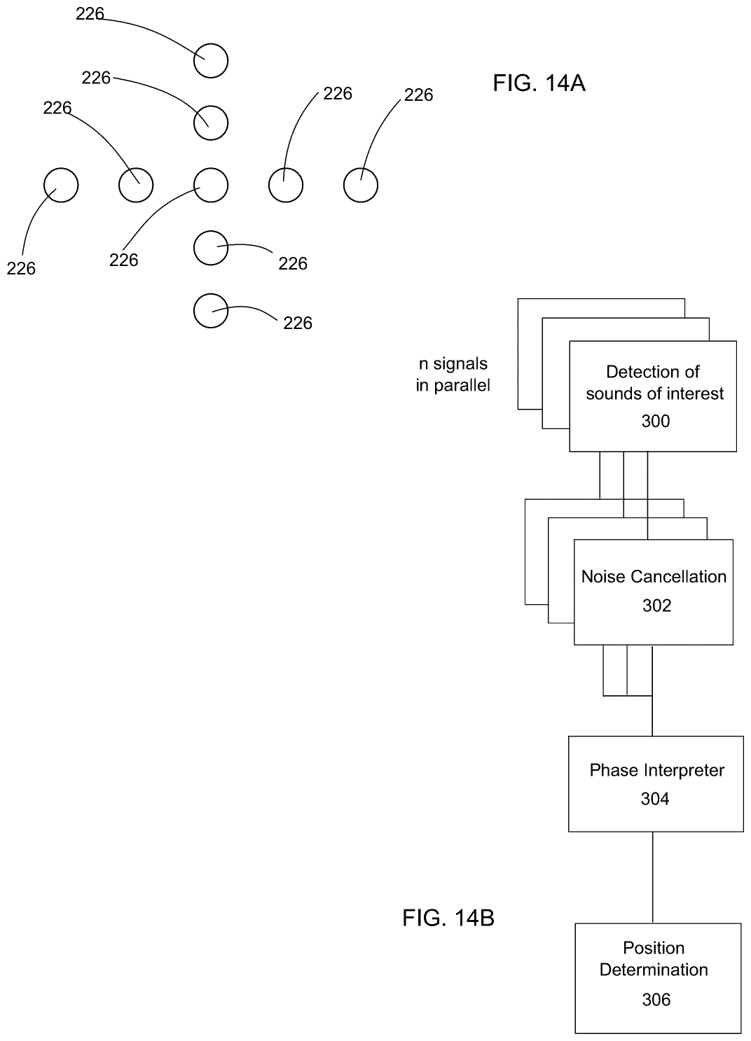

In some embodiments, the directional determination may also be carried out by arranging the communications device microphones 226 in a generally orthogonal configuration, such as that shown in FIG. 14A.

The process is depicted generally in FIG. 14B. At step 300, the input from the communications device microphones 226 may be examined (e.g. by the communications device processor 204) to detect possible particular sounds of interest. At step 302, noise cancellation may be applied by the communications device processor 204 to separate background noise from the particular sounds of interest (e.g. sirens, warning signals, etc.). The steps 300 and 302 may be conducted on a number of signals in parallel. For example, the number of signals may be equal to the number of the communications device microphones 226. At step 304, the communications device processor 204 may also apply phase interpreters to calculate the phase distribution of the sounds of interest among the different ones of the communications device microphones 226. Based on the phase and position of each of the communications device microphones 226, a position of the source of the sounds of interest may be calculated at step 306.

The communications device 202 is also able to estimate a distance and a trajectory of the source of the sound pattern using the magnitude of the fundamental frequency and harmonics of the pattern.

By way of example only, a particular sound of interest (e.g. a siren) may have a particular fundamental frequency with a magnitude M₀. There may be additional corresponding frequency bands above and below the fundamental frequency, with each of these frequency bands having magnitudes M₁, M₂, M₃, etc. (for bands that are above the fundamental frequency) and corresponding magnitudes M₋₁, M₋₂, M₋₃, etc. (for bands that are below the fundamental frequency. The ratios (M₁/M₀)=a₁, (M₋₁/M₀)=a₋₁, etc. may be determined. The difference (a₁−a₋₁)=b₀ may then be determined.

As the source of the sound of interest moves, the apparent frequency of the sound of interest will change, due to Doppler shift, depending on whether the source of the sound of interest is moving closer or further away. This shift in the apparent frequency of the fundamental frequency (and in the additional corresponding bands above and below the fundamental frequency) may be detected. For example, if the source of the sound of interest is moving towards the communications device 202, the apparent frequency is increased.

In addition, the corresponding magnitudes (e.g. M₋₃′, M₋₂′, M₋₁′, M₀′, M₁′, M₂′, M₃′, etc.) will also differ from before, depending on whether the source of the sound of interest is moving closer or further away. For example, if the source of the sound of interest is moving towards the communications device 202, the magnitudes for some of the bands may increase while some may decrease.

New ratios a₁′ and a₋₁′ may be calculated. For example, a₁′ may be expressed as follows:

$${\sum }_{i = 1}^{k} \frac{{M}_{i}^{′}}{{M}_{0}^{′}} , where k = number of harmonics compared$$

Similarly, a₋₁ may be expressed as follows:

$${\sum }_{i = 1}^{k} \frac{{M}_{- i}^{′}}{{M}_{0}^{′}} , where k = number of harmonics compared$$

A new difference of b₀′ (=a₁′−a₋₁′) may be determined. If b₀′ is greater than b₀, this is an indication that the source of the sound of interest is moving towards the communications device 202. Conversely, if b₀′ is less than b₀, this is an indication that the source of the sound of interest is moving away from the communications device 202. By using the above techniques, it is also possible to estimate a speed at which the source of the sound of interest is moving towards or away from the communications device 202.

The communications device 202 may be configured to only notify the user 208 when the

source of the sound of interest is approaching and is within a certain threshold distance. The speed of the source of the sound of interest may also be taken into account. For example, the estimated speed may be used to adjust the detection sensitivity versus a distance of the source of the sound of interest.

It is possible to calibrate the communications device 202 based on the fundamental frequency of the sound of interest at a given speed of approaching to or departing from the user. Once the calibration is completed, this can be shared with other ones of the communications devices 202 in the same area. It is possible to monitor the frequency bands below and above the fundamental frequency and, by using a differential ratio of magnitudes of these bands to fundamental frequency band due to Doppler shift, estimate a speed of the source of the sound of interest.

In some embodiments, the communications device 202 may be used to detect jet engine sound, and based on the frequency of the sound, to detect the throttle level of the jet engine. Based on the amplitude of the sound, the communications device 202 may be used to determine an approximate distance of the communications device 202 from the jet engine. By determining the throttle level and a distance to the jet engine, the communications device 202 may be configured to alert the user 208 when the user 208 is too close to the live engine (e.g. within a certain threshold distance). The detection of jet engine sound will not interfere with the performance of other functionalities of the communications device 202 and can be run while communication (described later) is in process. Higher throttle levels in jet engines generate higher frequency noise. The communications device 202 is able to map the frequency generated by jet engines to their throttle level (which may be different for different engine models), and based on that, calculate the throttle level. The distance to a jet engine can be calculated based on both throttle level and the sound level. Higher throttle levels generate more intense sounds, which the communications device 202 can take into account.

In some embodiments, the communications device 202 may be used to transmit and relay voice communications among the users of the communications device 202. For example, the communications device 202 may use BLE-based communications, depending on the distance among the communications device 202. The communications device 202 may also be configured to relay communications between other ones of the communications devices 202, as described further below.

In some embodiments, voice may be captured by the communications device 202 (e.g. through communications device microphones 226 directly or some other external audio input), and noise cancellation and coding can be applied to it by the communications device processor 204. The resultant data may be sent to the central gateway 54 to which other ones of the communications devices 202 are also connected. If other ones of the communications devices 202 are set to ‘listen” to a particular channel or a particular one of the communications devices 202, the other ones of the communications devices 202 would be able to hear sound (through the communications device speakers 228 directly or some other audio output, such as through headphones or earphones) from the particular channel or from the particular one of the communications devices 202. This type of voice communication has potentially unlimited range (referred to as “URPTT”) and may be two-way or one-way. This type of voice communication may use one or more of Internet, Wi-Fi, Internet of Things, or Bluetooth to connect the communications devices 202.

In some embodiments, the voice communication can be transferred between two or more of the communications devices 202. This may be used for relatively short-range voice communications (referred to as “SRPTT”). Voice will be recorded by the communications device 202 (such as using the communications device microphones 226), and potential noise cancellation and coding algorithms may be applied. The resultant data will be sent to other ones of the communications devices 202 that have been previously paired. Such communications may be transmitted through one or more of BLE, WIFI, or Bluetooth.

The communications devices 202 may comprise one or more communications device buttons 230 that may be depressed or otherwise activated by the user 208 to initiate a voice conversation with other ones of the communications device 202. The communications device buttons 230 may be referred to as “PTT” or “Push-to-Talk”.

In some embodiments, to start either the URPTT or SRPTT process, the communications device button 230 may be pressed and held by the user 208. While the user 208 is holding down the communications device button 230, the user 208 can talk and his or her speech will be recorded by the communications device microphones 226 and transmitted to the other ones of the communications devices 202. In some other embodiments, the user 208 may push the communications device button 230 a number of times in rapid succession (e.g. twice or thrice) in order to start recording by the communications device 202 (i.e. to start a conversation). In still some other embodiments, the user 208 is not required to hold down the communications device button 230 to initiate the conversation. This may be referred as hands-free PTT. In some embodiments, in order to terminate the conversation, the user 208 may press the communications device button 230.

Both SRPTT and URPTT processes may enable communication among communications devices 202 that are “registered” with the same organization.

In some embodiments, to make the SRPTT and URPTT processes more secure, each of the communications devices 202 must first be registered. The registration process may be conducted by one of the communications devices 202 connecting to another one of the communications device 202 (e.g. using BLE connectivity). In some embodiments, the user 208 may be prompted to create a password for each of the communications devices 202 or security can be enabled by face ID, biometric, or other identifying technology.

In some embodiments, the SRPTT process will always connect nearby ones of the users 208 that are carrying the communications devices 202. In addition, the communications devices 202 may continue to refresh to search for the new ones of the communications devices 202 that are within range. It is possible to customize the range in which the SRPTT process is used to connect nearby ones of the communications devices 202.

The SRPTT process may use different techniques to estimate the proximity of other ones of the communications devices 202. Such techniques may be through using Bluetooth, Wi-Fi, or the like, where lower signal strength can represent users farther away. For example, the communications device processor 204 may be configured to have access to information regarding signal strength from external sources (e.g., a signal strength meter IC) or using a RSSI index within the communications devices 202.

In some embodiments, one of the communications devices 202 (e.g. 202a) may connect to another one of the communications devices 202 (e.g. 202b) using some electromagnetic-based communications standard (e.g. Bluetooth, Bluetooth Low Energy, Zigbee, or the like). The communications device 202a may also be configured to send a pattern of sound in the form of a short pulse of sounds containing one or more frequency components to the communications device 202b. A distance between the communications device 202a and the communications device 202b may be estimated as follows. At or around the time the pulse of sound is transmitted by the communications device 202a, the communications devices 202b is also configured to transmit (using an electromagnetic-based communications standard) a time stamp comprising the time the sound pulse is transmitted. It is also possible to calculate and take into account the delay that it takes to generate the sound pulse into the time stamp. When the communications device 202b receives the sound pulse, the communications device 202b is able to determine the time at which the pulse is received. By comparing the time at which the pulse is received with the time in the time stamp (indicating when the pulse was transmitted), and based on the speed of sound in air, the communications device 202b is able to estimate the distance between the communications device 202a and the communications device 202b.

In another embodiment, the communications device 202a is configured to record the time that the sound pulse is transmitted. When the communications device 202b receives the sound pulse, the communications device 202b is configured to transmit an acknowledgment to the communications device 202a (using an electromagnetic-based communications standard). When the communications device 202a receives the acknowledgment, the communications device 202a is configured to compare the time at which the acknowledgement is received with the time at which the sound pulse is transmitted. The difference between the two times is the time of flight (of the sound pulse). Using this time of flight, it is possible for the communications device 202a to estimate the distance between the communications device 202a and the communications device 202b. This approach does not require that the communications devices 202a, 202b run on synchronous clocks.

It is also possible to implement the above distance estimation methods using other devices other than the communications devices 202. For example, it is possible to use a first handheld mobile device that is configured to generate sound and that is configured to communicate using an electromagnetic-based communications standard (e.g. Bluetooth, Bluetooth Low Energy, Zigbee, or the like). By also using one or more second handheld mobile devices that are configured to receive sound (e.g. through a microphone) and that are configured to communicate using the electromagnetic-based communications standard, the first handheld mobile device is able to estimate a distance between it and one or more of the second handheld mobile devices using the methods discussed above.

In some embodiments, the SRPTT process may only connect to a limited number of communications devices 202. The SRPTT process may use a group identification tag and/or estimated distances among the communications devices 202 as factors for choosing which limited ones of the communications devices 202 would be selected to be connected. For example, the SRPTT process may connect the communications device 202 to the nearest five other ones of the communications devices 202 that share the same particular group identification tag. The group identification tag may be set during registration.

To ensure that the SRPTT and URPTT processes and streaming have substantially maximum quality of sound, the communications device processor 204 may be configured to execute a programmable coder and decoder algorithm that adjusts the data compression of the raw signals. When the number of connected communications devices 202 increases or the communications devices 202 are located farther away (i.e. more lossy channel), the communications device processor 204 may be configured to increase the compression ratio, and when number of communications devices 202 decreases or the distances are shortened, the communications device processor 204 may be configured to use lower compression ratio and higher sound quality.

In some embodiments, during registration of each of the communications devices 202, a software tag may be generated and assigned to the communications devices 202 belonging to the same group of employees or affiliated users to identify them from each other. The software tag may be advertised by the communications device 202 after SRPTT and URPTT activation, and the communications device 202 may be able to listen to and connect by SRPTT to other ones of the communications devices 202 carrying the similar tag or tags. This is useful to ensure that the SRPTT process is only functioning for the same organization's employees or for particular groups inside the organization.

The communications device 202 may allow the user 208 to select from a number of voice channels that the user 208 can choose to listen to. When the user 208 selects one or more of the channels, the user 208 is notified about an incoming conversation if the user 208 is not carrying the communications device 202 or if the communications device 202 is otherwise disconnected. For example, the communications device 202 may be connected to the mobile device 60. This notification can be in the form of an alarm and/or a written notification message to the mobile device 60.

In some embodiments, if the user 208 is carrying the communications device 202 and chooses to listen to one or multiple ones of the channels/groups, when an incoming conversation arrives in any of these channels, the user 208 can listen to them. If the user 208 choses to reply, the user 208 can press the communications device button 230, and the communications device 202 will automatically send the vocal response of the user 208 to the last channel from which the user 208 received conversation. Alternatively, the communications device 202 may be configured such that if the user 208 presses the communications device button 230 within a certain amount of time, the communications device 202 will send the response of the user 208 to the last channel from which the user 208 received conversation. If the user presses the communications device button 230 after a certain amount of time, the communications device 202 will send the response of the user 208 to a preprogrammed channel.

In some embodiment, the communications device 202 may be configured such that the communications device button 230 does not need to be pressed to initiate conversations. In such embodiment, the communications device 202 may be configured to detect voice (i.e. from a person talking). If the communication device 202 detects one or more voices, the communications device 202 may be configured to automatically commence a conversation.

In some embodiments, the communications devices 202 may be configured to transmit to other ones of the communications devices 202 using the BLE protocol when the communications devices 202 are within range of the BLE protocol. When the communications devices 202 are beyond the range of the BLE protocol, the communications devices 202 may be configured to use one or more of radio communications, Wi-Fi communications, and cellular network protocols to communicate with other ones of the communications devices 202. When the communications devices 202 utilize radio communications, Wi-Fi communications, or cellular networks, the communications devices 202 may connect through the server 7.

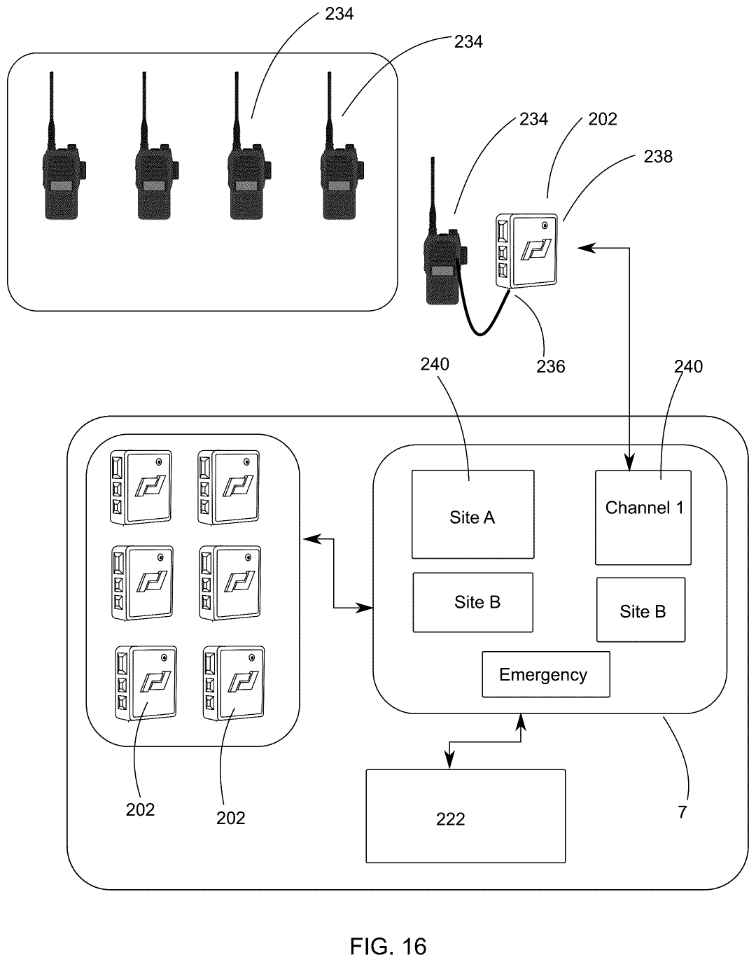

Referring to FIG. 16, one of the communications devices 202 may be configured to act as a relay 238 between conventional radio communications and the communications described above (e.g. SRPTT and/or URPTT). For example, a plurality of radios 234 may be communicating with each other using conventional radio communications. By connecting one of the radios 234 to the relay 238, such as through an input port 236 on the relay 238, it is possible to relay the audio of the radio communications to the server 7. The server 7 may be configured to allow other ones of the communications device 202 to access the audio of the radio communications and to transmit audio. For example, referring to FIG. 16, the plurality of radios 234 may be communicating among each other using a conventional radio channel (e.g. Radio Ch-1). The audio from Radio Ch-1 may be relayed by the relay 238 to the server 7, where other ones of the communications devices 202 are able to access the audio, depending on whether they are authorized or assigned to access the audio. The other ones of the communications devices 202 may also transmit audio to the server 7, which in turn would be relayed by the relay 238 to be transmitted by the radios 234 using conventional radio communications. In this manner, it is possible for one of the users 208 (using one of the communications devices 202) to remotely participate in audio conversations with persons using the radios 234, even if the user 208 would be otherwise out of normal radio communications range.

As shown in FIG. 16, there may be a plurality of channels 240 maintained by the server 7. The interface 222 may be used to access the server 7 in order to graphically depict information regarding the communications devices 202, including the approximate locations of the communications devices 202. In addition, the interface 222 may be used to manage and connect to the different ones of the channels 240. Furthermore, the interface 222 may be used to assign tasks to one or more of the users 208 (including, but not limited to, those users using the radios 234 and those users using SRPTT and/or URPTT). The interface 222 may also be used to access (e.g. listen and/or talk to the radios 234) the channels 240, including those that are conventional radio channels.

The communications device 202 and/or the server 7 may be configured to manage communication based on priorities. For example, connections may be made to prioritize communication for pedestrian safety. Inactive ones of the communications devices 202 that do not need to communicate with each other based on their pedestrian safety setting and PTT setting (e.g. either SRPTT or URPTT) will not connect to each other. In this manner, connection bandwidth and availability will be more reserved.

By way of example only, low priority connections will allow communications devices 202 to connect and communicate if higher priority ones of communications devices 202 are not nearby. Communications devices 202 with low priority connections will be disconnected if higher priority ones of the communications devices 202 enter the area.

A connection between communications devices 202 may be considered high priority if, for example, pedestrian safety is enabled for one or more of the communications devices 202. A connection between communications devices 202 may be considered low priority if, for example, pedestrian safety is disabled for one or more of the communications devices 202. Alternatively, a connection between communications devices 202 may be considered low priority if one or more of the communications devices 202 is currently using radio communications (rather than BLE-based communications) or the communications devices 202 is being charged and/or not used by a driver of the vehicle 210 on which the communications device 202 is located.

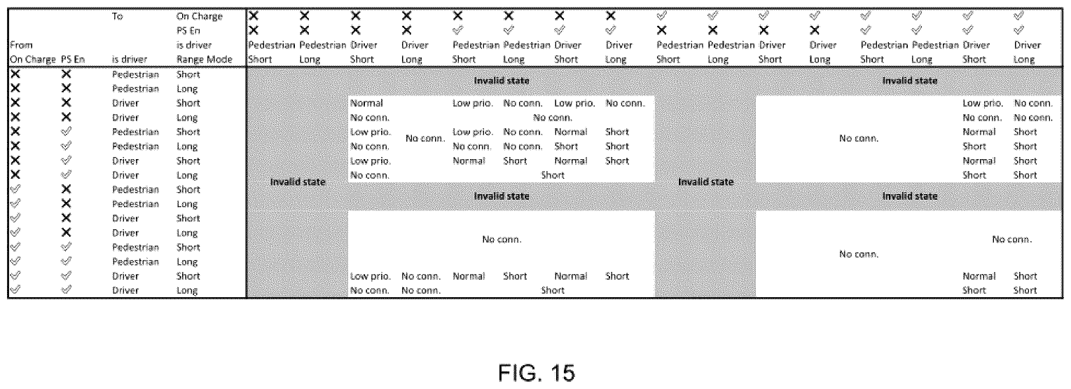

FIG. 15 is a table depicting one possible management of the priorities, such as by the server 7. The top four rows of FIG. 14 indicate the possible states for the communications device 202 that is receiving a message, while the first four columns of FIG. 14 indicate the possible states for the communications devices 202 that is sending the message. For example, the possible states include (1) “On Charge”, which is an indication if the communications device 202 is currently being charged; (2) “PS En”, which is an indication if the communications device 202 has the pedestrian safety mode engaged; (3) “is driver” , which is an indication if the communications device 202 is located on the vehicle 210 (i.e. a driver) or on the user 208 (i.e. a pedestrian); and (4) “Range Mode”, which is an indication if the communications devices 202 is currently using short-range communications (SRPTT) or longer-range communications (URPTT). Pedestrian safety mode refers to some of the pedestrian safety features described herein (e.g. for detecting when the user 208 carrying the communications device 202 is determined to be close to the vehicle 210 with the communications device 202 installed). The pedestrian safety mode may be set for each of the communications devices 202.

The values within the table of FIG. 15 denote the relative priorities of the connections. If the communications device 202 is being charged, it may be assumed that the communications device 202 is not moving. If the value shown in the table is indicated as “Short”, this indicates that short-range communications (e.g. using BLE) will be prioritized between the communications devices 202. This is because in some embodiments, the number of “short-range” communications connections is limited, and priority is given to certain types of connections, as set out in the Table.

If the value is shown as “Normal”, this indicates that no special priority will be given. If the value is shown as “No conn.”, this indicates that no connection between the communications devices 202 will be established.

For connections with values shown as “Low prio.” (or low priority), the connection may be maintained for communication, but such connections may be disconnected if more communications devices 202 with higher priority connections are nearby or present.

As discussed above, the communications devices 202 may use BLE-based communications to exchange audio data (such as voice communications). As the distance between communications devices 202 increases, the signal strength decreases, and the likelihood of BLE packets failing the CRC check increases. When that happens, the communications devices 202 may retransmit the packet. Also, as the number of connection increase, the time that the connection can utilize reduces due to the other connections. Hence the number of times the communications devices 202 can transmit is reduced. If the communications devices 202 fails to send the packet on time, it will result in a delay in audio or cuts in the audio. Based on the RSSI strength of the connection and the number of other connections, the communications device 202 is configured to adjust the sampling rate of the audio being encoded, adjusting the encoded frame size, to do one of the following:
(1) If the RSSI is low or the number of connections is higher than a set threshold, the communication device 202 is configured to decrease the sampling rate (reducing the encoded frame size), pack longer audio in a packet with the same size, and/or giving the communication device 202 more time to retry before resulting in cuts, in expense of lower audio quality.
(2) If the RSSI is low or the number of connections is higher than a set threshold, the communications device 202 is configured to decrease the sampling rate (reducing the encoded frame size) and/or pack the same amount of audio with a smaller size packet (reducing the chance of packet corruption and increasing the chance of packet is successfully delivered), in expense of lower audio quality.
(3) If the RSSI is high and the number of connections is lower than a set threshold, the communications device 202 is configured to increase the sampling rate (increasing the encoded frame size), pack less audio in the same size, and/or increase the quality of the audio.

The above may be implemented for all connected ones of the communications devices 202 if the communications device 202 sending lacks computational resources and can only encode once. Alternatively, if the communications device 202 has more computational resources, it could resample and encode separately for different connection conditions, given the number of connections is lower than the set threshold.

The communications devices 202 may need to exchange the sampling rate that the encoded audio is originally in so that it may be correctly decoded. This information may be exchanged using one or more flags in the packet exchanged in order to change the decode and playback to the correct settings.

In the foregoing description, exemplary modes for carrying out the invention in terms of examples have been described. However, the scope of the claims should not be limited by those examples but should be given the broadest interpretation consistent with the description as a whole. The specification and drawings are, accordingly, to be regarded in an illustrative rather than a restrictive sense.

1. A safety system comprising:
a plurality of communications devices, each of the communications devices configured to be in communication with one or more of other ones of the communications devices, wherein each of the communications devices comprises a transceiver;
wherein each of the communications devices is adapted to determine an approximate distance to the one or more of the other ones of the communications devices and to generate a warning when one of the approximate distances is less than a predetermined distance; and
wherein a determination of the approximate distance to the one or more of the other ones of the communications devices is based, at least in part, on one or both of received signal strength indicator (RSSI) or global positioning system (GPS), depending on the predetermined distance.

2. The safety system of claim 1, wherein the communications device is further adapted to use machine learning techniques and RSSI to determine the approximate distance.

3. The safety system of claim 1, wherein one or more of the communications devices are carried by users and one or more of the communications devices are configured to be mounted in vehicles.

4. The safety system of claim 3, wherein the vehicles comprise a display configured to display a relative location of other ones of the communications devices.

5. The safety system of claim 4, further comprising a server, wherein the server is configured to communicate with one or more of the communications devices and to receive from the one or more of the communications devices a respective location of each of the one or more of the communications devices.

6. The safety system of claim 5, further comprising a mobile device and a code, wherein the code is adapted to be scanned by the mobile device, and wherein upon scanning of the code by the mobile device, the mobile device is configured to transmit to the server a location of the mobile device, wherein the server is configured to generate the warning when the mobile device is less than the predetermined distance to one of the communications devices.

7. The safety system of claim 5, further comprising a mobile device and a code, wherein the code is adapted to be scanned by the mobile device, and wherein upon scanning of the code by the mobile device, the mobile device is configured to determine, based, at least in part, on the power of signals received from the mobile device from one or more of the communications devices, an approximate distance to the one or more of the communications devices and to generate a warning when one of the approximate distances is less than a predetermined distance.

8. A communications device for use in a vehicle with a plurality of speakers, the communications device comprising:
a plurality of microphones adapted to convert acoustic signals into electric signals, wherein the plurality of microphones are located on various locations on the vehicle;
a processing unit coupled to the microphones, wherein the processing unit is configured to receive the electrical signals from the plurality of microphones, wherein the processing unit is further configured to compare first parameters of the electrical signals received from the microphones with second parameters of predetermined sounds of interest to determine whether the electrical signals comprise one or more of the predetermined sounds of interest and a direction from which the acoustic signals are originating, and wherein upon the processing units determines that the electrical signals comprise one or more of the predetermined sounds of interest, the processing unit is configured to cause one or more of the speakers to play the predetermined sounds of interest, the processing unit selecting the particular ones of the one or more of the speakers to provide directionality to the predetermined sounds of interest.

9. The communications device of claim 8, wherein the processing unit is further configured to determine whether a source of the predetermined sounds of interest is moving away from or towards the communications device by comparing differences between relative magnitudes of a fundamental frequency and associated frequency bands of the predetermined sounds of interest over time.

10. The communications device of claim 8, wherein the predetermined sounds of interest correspond to jet engine sounds.

11. A system for communications, the system comprising:
a server;
one or more communications devices, each of the communications devices comprising:
audio input and output; and
a transceiver configured to communicate with the server and with other ones of the communications devices;
wherein the communications devices are configured to communicate with other ones of the communications devices either (a) directly, when the communications devices are within a certain distance of each other, or (b) through the server.

12. The system of claim 11, wherein the communications devices are configured to allow for selection between communicating with other ones of the communications devices directly or through the server.

13. The system of claim 11, wherein the communications devices are configured to communicate with other ones of the communications devices directly using Bluetooth Low Energy (BLE) when the communications devices are within the certain distance of each other.

14. The system of claim 11, wherein the communications devices are configured to communicate with other ones of the communications devices through the server using one or both of cellular networks or Wi-Fi.

15. The system of claim 11, further comprising one or more radio transmitters in radio communications with each other, wherein one or more of the radio transmitters are coupled to one or more of the communications devices to relay communications among the radio transmitters and other ones of the communications devices.

16. The system of claim 15, wherein the one or more radio transmitters and the one or more communications devices are grouped into one or more channels, wherein communications are limited to the one or more radio transmitters and the one or more communications devices within each of the channels.

17. The system of claim 16, wherein the server comprises an interface, the interface configured to allow for access to communications within different ones of the channels.

18. The system of claim 17, wherein the server is configured to prioritize certain ones of the connections between two of the communication devices.

19. The system of claim 18, wherein the server is configured to prioritize certain ones of the connections, depending, at least in part, on a charging status of the communications devices, an installation location of the communications devices, and a current status of the communications between the communication devices.
